SEQUENCE LISTING (55 sequences)

SEQ ID NO: 1 - protein, 188 aa, Homo Sapiens
MDCRKMARFSYSVIWIMAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 2 - protein, 188 aa, Homo Sapiens
MDCRKMVRFSYSVIWIMAISKAFELGLVAGLGHQEFARPSRGDLAFRDDSIWPQEEPAIRPRSSQRVLPMGIQHSKELNRTCCLNGGTCMLESFCACPPSFYGRNCEHDVRKENCGSVPHDTWLPKKCSLCKCWHGQLRCFPQAFLPGCDGLVMDEHLVASRTPELPPSARTTTFMLAGICLSIQSYY

SEQ ID NO: 3 - DNA, 593 bp, Homo Sapiens
ggatccttcaatatggactgcaggaagatggcccgcttctcttacagtgtgatttggatcatggccatttctaaagtctttgaactgggattagttgccgggctgggccatcaggaatttgctcgtccatctcggggatacctggccttcagagatgacagcatttggccccaggaggagcctgcaattcggcctcggtcttcccagcgtgtgccgcccatggggatacagcacagtaaggagctaaacagaacctgctgcctgaatgggggaacctgcatgctggggtccttttgtgcctgccctccctccttctacggacggaactgtgagcacgatgtgcgcaaagagaactgtgggtctgtgccccatgacacctggctgcccaagaagtgttccctgtgtaaatgctggcacggtcagctccgctgctttcctcaggcatttctacccggctgtgatggccttgtgatggatgagcacctcgtggcttccaggactccagaactaccaccgtctgcacgtactaccacttttatgctagttggcatctgcctttctatacaaagctactattaagcggccgcctcgag

SEQ ID NO: 4 - DNA, 567 bp, Homo Sapiens
atggactgcaggaagatggtccgcttctcttacagtgtgatttggatcatggccatttctaaagcctttgaactgggattagttgccgggctgggccatcaggaatttgctcgtccatctcggggagacctggccttcagagatgacagcatttggccccaggaggagcctgcaattcggcctcggtcttcccagcgtgtgctgcccatgggaatacagcacagtaaggagctaaacagaacctgctgcctgaatgggggaacctgcatgctggagtccttttgtgcctgccctccctccttctacggacggaactgtgagcacgatgtgcgcaaagagaactgtgggtctgtgccccatgacacctggctgcccaagaagtgttccctgtgtaaatgctggcacggtcagctccgctgctttcctcaggcatttctacccggctgtgatggccttgtgatggatgagcacctcgtggcttccaggactccagaactaccaccgtctgcacgtactaccacttttatgctagctggcatctgcctttctatacaaagctactattaa

SEQ ID NO: 5 - DNA, 26 bp, Artificial Sequence
  Synthetic oligonucleotide
gagaagcggaccatcttcctgcagtc

SEQ ID NO: 6 - DNA, 25 bp, Artificial Sequence
  Synthetic oligonucleotide
agagaagcggaccatcttcctgcag

SEQ ID NO: 7 - DNA, 25 bp, Artificial Sequence
  Synthetic oligonucleotide
taagagaagcggaccatcttcctgc

SEQ ID NO: 8 - DNA, 25 bp, Artificial Sequence
  Synthetic oligonucleotide
gtattcccatgggcagcacacgctg

SEQ ID NO: 9 - DNA, 25 bp, Artificial Sequence
  Synthetic oligonucleotide
ctgtattcccatgggcagcacacgc

SEQ ID NO: 10 - DNA, 25 bp, Artificial Sequence
  Synthetic oligonucleotide
gctgtattcccatgggcagcacacg

SEQ ID NO: 11 - DNA, 25 bp, Artificial Sequence
  Synthetic oligonucleotide
ggcacaaaaggactccagcatgcag

SEQ ID NO: 12 - DNA, 25 bp, Artificial Sequence
  Synthetic oligonucleotide
caggcacaaaaggactccagcatgc

SEQ ID NO: 13 - DNA, 25 bp, Artificial Sequence
  Synthetic oligonucleotide
tatagaaaggcagatgccagctagc

SEQ ID NO: 14 - DNA, 25 bp, Artificial Sequence
  Synthetic oligonucleotide
cgggtcatgaaatttgcataatatc

SEQ ID NO: 15 - DNA, 25 bp, Artificial Sequence
  Synthetic oligonucleotide
tacgggtcatgaaatttgcataata

SEQ ID NO: 16 - DNA, 26 bp, Artificial Sequence
  Synthetic oligonucleotide
gagaagcgggccatcttcctgcagtc

SEQ ID NO: 17 - DNA, 25 bp, Artificial Sequence
  Synthetic oligonucleotide
agagaagcgggccatcttcctgcag

SEQ ID NO: 18 - DNA, 25 bp, Artificial Sequence
  Synthetic oligonucleotide
taagagaagcgggccatcttcctgc

SEQ ID NO: 19 - DNA, 25 bp, Artificial Sequence
  Synthetic oligonucleotide
gtatccccatgggcggcacacgctg

SEQ ID NO: 20 - DNA, 26 bp, Artificial Sequence
  Synthetic oligonucleotide
ctgtatccccatgggcggcacacgct

SEQ ID NO: 21 - DNA, 25 bp, Artificial Sequence
  Synthetic oligonucleotide
gctgtatccccatgggcggcacacg

SEQ ID NO: 22 - DNA, 25 bp, Artificial Sequence
  Synthetic oligonucleotide
ggcacaaaaggaccccagcatgcag

SEQ ID NO: 23 - DNA, 25 bp, Artificial Sequence
  Synthetic oligonucleotide
caggcacaaaaggaccccagcatgc

SEQ ID NO: 24 - DNA, 25 bp, Artificial Sequence
  Synthetic oligonucleotide
tatagaaaggcagatgccaactagc

SEQ ID NO: 25 - DNA, 25 bp, Artificial Sequence
  Synthetic oligonucleotide
ctggtcatgaaatttgcatgatatc

SEQ ID NO: 26 - DNA, 25 bp, Artificial Sequence
  Synthetic oligonucleotide
tactggtcatgaaatttgcatgata

SEQ ID NO: 27 - DNA, 19 bp, Artificial Sequence
  Synthetic oligonucleotide
gcgtgtgctgcccatggga

SEQ ID NO: 28 - DNA, 20 bp, Artificial Sequence
  Synthetic oligonucleotide
cgggtcatgaaatttgcata

SEQ ID NO: 29 - DNA, 24 bp, Artificial Sequence
  Synthetic oligonucleotide
gactgcaggaagatggtccgcttc

SEQ ID NO: 30 - DNA, 21 bp, Artificial Sequence
  Synthetic oligonucleotide
ttcccatgggcagcacacgct

SEQ ID NO: 31 - DNA, 23 bp, Artificial Sequence
  Synthetic oligonucleotide
gcgtgtgctgcccatgggaatac

SEQ ID NO: 32 - DNA, 21 bp, Artificial Sequence
  Synthetic oligonucleotide
gcaggcacaaaaggactccag

SEQ ID NO: 33 - DNA, 23 bp, Artificial Sequence
  Synthetic oligonucleotide
gcagatgccagctagcataaaag

SEQ ID NO: 34 - DNA, 21 bp, Artificial Sequence
  Synthetic oligonucleotide
gctagctggcatctgcctttc

SEQ ID NO: 35 - DNA, 22 bp, Artificial Sequence
  Synthetic oligonucleotide
acgggtcatgaaatttgcataa

SEQ ID NO: 36 - DNA, 23 bp, Artificial Sequence
  Synthetic oligonucleotide
gctacgaccttctggggaaaacg

SEQ ID NO: 37 - DNA, 20 bp, Artificial Sequence
  Synthetic oligonucleotide
ctggtcatgaaatttgcatg

SEQ ID NO: 38 - DNA, 24 bp, Artificial Sequence
  Synthetic oligonucleotide
gactgcaggaagatggcccgcttc

SEQ ID NO: 39 - DNA, 21 bp, Artificial Sequence
  Synthetic oligonucleotide
tccccatgggcggcacacgct

SEQ ID NO: 40 - DNA, 23 bp, Artificial Sequence
  Synthetic oligonucleotide
gcgtgtgccgcccatggggatac

SEQ ID NO: 41 - DNA, 21 bp, Artificial Sequence
  Synthetic oligonucleotide
gcaggcacaaaaggaccccag

SEQ ID NO: 42 - DNA, 23 bp, Artificial Sequence
  Synthetic oligonucleotide
gcagatgccaactagcataaaag

SEQ ID NO: 43 - DNA, 21 bp, Artificial Sequence
  Synthetic oligonucleotide
gctagttggcatctgcctttc

SEQ ID NO: 44 - DNA, 21 bp, Artificial Sequence
  Synthetic oligonucleotide
ctggtcatgaaatttgcatga

SEQ ID NO: 45 - DNA, 22 bp, Artificial Sequence
  Synthetic oligonucleotide
cgcttacaggaattgcccttgc

SEQ ID NO: 46 - DNA, 20 bp, Artificial Sequence
  Synthetic oligonucleotide
cagacccaagctatcgcagc

SEQ ID NO: 47 - DNA, 21 bp, Artificial Sequence
  Synthetic oligonucleotide
ggctgagtctccagctcaagg

SEQ ID NO: 48 - DNA, 26 bp, Artificial Sequence
  Synthetic oligonucleotide
gtatttctggaaataggtcaatgtcg

SEQ ID NO: 49 - DNA, 20 bp, Artificial Sequence
  Synthetic oligonucleotide
tgtgatttggatcatggcca

SEQ ID NO: 50 - DNA, 21 bp, Artificial Sequence
  Synthetic oligonucleotide
ctcatgtttgacttcctcttc

SEQ ID NO: 51 - DNA, 23 bp, Artificial Sequence
  Synthetic oligonucleotide
catcgaagtcaggcagttcttac

SEQ ID NO: 52 - DNA, 20 bp, Artificial Sequence
  Synthetic oligonucleotide
gatcatggccatttctaaag

SEQ ID NO: 53 - DNA, 24 bp, Artificial Sequence
  Synthetic oligonucleotide
gaatttgctcgtccatctcgggga

SEQ ID NO: 54 - DNA, 17 bp, Artificial Sequence
  Synthetic oligonucleotide
taattctaccaaggtct

SEQ ID NO: 55 - DNA, 10 bp, Artificial Sequence
  Synthetic oligonucleotide
ctcttcagaa

(19) United States
(12) Patent Application Publication
(10) Pub. No.: US 2010/0041032 A1
(43) Pub. Date: Feb. 18, 2010

(54) COMPOSITION AND METHODS FOR THE DETECTION OF CRIPTO-3

(75) Inventors: Olivia Orozco, Arlington, MA (US); Michele Sanicola-Nadel, Winchester, MA (US); Eugene Choi, Somerville, MA (US); John Carulli, Southborough, MA (US); Richard Tizard, Framingham, MA (US); Suzanne Szak, Arlington, MA (US); Chao Sun, Needham, MA (US)

Correspondence Address: LAHIVE & COCKFIELD, LLP / BIOGEN IDEC;FLOOR 30, SUITE 3000, ONE POST OFFICE SQUARE, BOSTON, MA, 02109-2127, US

(73) Assignee: Biogen Idec MA Inc., Cambridge, MA (US)

(21) Appl. No.: 12/298,875

(22) PCT Filed: Apr. 30, 2007
(86) PCT No.: PCT/US07/10399

§ 371 Date: Oct. 13, 2009

Related U.S. Application Data
(60) Provisional application No. 60/795,807, filed on Apr. 28, 2006.

(51) Int. Cl. C12Q 1/68; C12P 21/08; C07K 16/00; C07H 21/04

(52) U.S. Cl. 435/6; 435/69.6; 530/388.1; 536/23.1

(57) ABSTRACT

The present invention is based, at least in part, on the discovery that the pseudogene TDGF3 (Cripto-3) is expressed in cells and, in particular, that TDGF3 overexpression is associated with transformation of a cell, e.g., TDGF3 is overexpressed in cancer cell lines and cells from tumor tissue. Accordingly, the invention provides compositions, kits, and methods for detecting the presence of a TDGF3 polynucleotide or polypeptide in a sample. The invention further provides compositions, kits and methods for assessing whether a cell is transformed as well as for assessing whether a patient is a suitable candidate for an anti-Cripto antibody therapy.

### RELATED APPLICATIONS

This patent application claims the benefit of U.S. Provisional Patent Application Ser. No. 60/795,807, entitled “Compositions and Methods for the Detection of Cripto-3”, filed Apr. 28, 2006. The entire contents of the above-referenced provisional patent application are incorporated herein by this reference.

### BACKGROUND OF THE INVENTION

Cripto-1 (encoded by TDGF1) is a cell surface-associated protein which contains one domain having similarity to transforming growth factor alpha and epidermal growth factor. The EGF-CFC family protein plays important roles in early development and cancer formation (See reviews: Gritsman et al. 1999; Minchiotti et al. 2001; Saloman et al. 2000; Strizzi et al. 2005)). A loss of function mutation in Cripto is associated with holoprosencphaly in humans, including forebrain defects, and developmental delay (de la Cruz et al. 2002). Expression of Cripto protein in normal adult tissues is low, and it is unclear if a function exists for this protein in normal adult tissue. One exception is in the mammary gland, where Cripto expression is suspected to play a role in ductal epithelial cell differentiation (Saloman et al. 2000). However, Cripto protein is overexpressed in many human solid tumors (Adkins et al. 2003; Ciardiello et al. 1991b; Shen 2003). For example, immunohistochemistry with anti-Cripto antibodies shows overexpression of Cripto in up to 80% of human breast tumors as well as a large proportion of colon and lung tumors. Cripto overexpression is also oncogenic (Sun Y 2005). It has also been shown that expression of Cripto leads to transformation of a normal mouse mammary epithelia cell line (Ciardiello et al. 1991a). Several recent publications have shown that inhibition of Cripto either by monoclonal antibodies or by antisense oligonucleotides inhibits cancer cell growth in vivo (Adkins et al. 2003; Normanno et al. 2004b; Xing et al. 2004).

While it is clear that Cripto is upregulated in many cancer cell lines and tumors, and that Cripto overexpression is oncogenic, it is less clear how Cripto expression is regulated in normal and cancer tissues. Moreover, it is not clear how many genes actually encode Cripto protein. There are at least seven CRIPTO genes and pseudogenes in the human genome, named as TDGF1 through TDGF7. TDGF1 is located on Chromosome 3 p23-21 region and is widely believed to be the only structural gene for Cripto protein (Table 1). Among the 6 pseudogenes, TDGF3 on the X chromosome (Xq28) has an intact open reading frame that could encode a predicted protein (Cripto-3) having six different amino acids as compared to the published Cripto-1 protein reference sequence (Scognamiglio B 1999) (FIG. 1A; SEQ ID NO:1). This gene is intronless, appears to be derived from an insertion of the TDGF1 cDNA into the human genome during evolution and, prior to the instant invention, was presumed to not be expressed. Accordingly there was a need in the art to examine the potential expression of the pseudogene TDGF3 and, further, any correlation that might exist between TDGF3 expression and the development or existence of a proliferative disorder such as cancer.

### SUMMARY OF THE INVENTION

The present invention is based, at least in part, on the discovery that the presumed pseudogene TDGF3 is a functional intronless gene expressed in human cells and, moreover, that TDGF3 overexpression is associated with transformation of a cell, e.g., TDGF3 is overexpressed in cancer cell lines and cells from tumor tissue. Accordingly, the invention relates to compositions, kits, and methods for specifically detecting the presence of a marker, e.g., TDGF3 and/or TDGF1, polynucleotide or polypeptide in a sample. These compositions, kits and methods are useful for determining the phenotype of a tumor, e.g., whether the tumor is a TDGF1 or TDGF3 expressing tumor. These compositions, kits and methods are further useful for assessing whether a cell is transformed, e.g., for diagnosing cancer, as well as for assessing whether a patient is a suitable candidate for an anti-Cripto antibody therapy. Accordingly, the invention further relates to compositions, kits and methods for determining the Cripto-expressing phenotype of a tumor. The invention further relates to compositions, kits and methods for assessing whether a cell is transformed. The invention further relates to compositions, kits and methods for assessing whether a patient is a suitable candidate for an anti-Cripto antibody therapy.

Accordingly, one aspect of the invention pertains to methods for detecting the presence of a TDGF3 polynucleotide or portion thereof in a sample, the method comprising the steps of:
a) contacting the sample with a nucleic acid molecule which selectively hybridizes to a transcribed TDGF3 polynucleotide, wherein the transcribed TDGF3 polynucleotide comprises the coding region of the TDGF3 gene; and
b) determining whether the nucleic acid molecule binds to the polynucleotide in the sample, to thereby detect the presence of the TDGF3 polynucleotide or portion thereof in the sample.

In one embodiment, the transcribed TDGF3 polynucleotide is mRNA.

In one embodiment, the transcribed TDGF3 polynucleotide is cDNA.

In one embodiment, the method further comprises the step of amplifying the transcribed TDGF3 polynucleotide with the nucleic acid molecule.

In one embodiment, the amplification step comprises a polymerase chain reaction.

In one embodiment, binding of the nucleic acid molecule to the transcribed TDGF3 polynucleotide is determined by detecting amplified TDGF3 polynucleotide.

In one embodiment, the transcribed polynucleotide is amplified using at least one nucleic acid molecule which selectively hybridizes to the transcribed TDGF3 polynucleotide.

In one embodiment, the at least one nucleic acid molecule does not amplify a TDGF1 polynucleotide.

In one embodiment, the at least one nucleic acid molecule hybridizes to a portion of the transcribed TDGF3 polynucleotide, which portion comprises nucleotides within the TDGF3 coding region encoding an amino acid selected from the group consisting of V7, L68, E92 and A178.

In one embodiment, the at least one nucleic acid molecule comprises a sequence selected from the group consisting of the sequences set forth in Table 2 and Table 3.

In one aspect, the invention pertains to a method for detecting the presence of a TDGF3 polypeptide or portion thereof in a sample, the method comprising the steps of:
a) contacting the sample with a reagent which selectively binds to a TDGF3 polypeptide; and
b) determining whether the reagent binds to the polypeptide in the sample, to thereby detect the presence of the TDGF3 polypeptide or portion thereof in the sample.

In one embodiment, the reagent is selected from the group consisting of an antibody, antibody derivative and an antibody fragment.

In one embodiment, the antibody, antibody derivative or antibody fragment binds to the TDGF3 polypeptide and does not bind to a TDGF1 polypeptide.

In one embodiment, the antibody, antibody derivative or antibody fragment binds to an epitope comprised in the extracellular portion of the TDGF3 polypeptide.

In one embodiment, the antibody, antibody derivative or antibody fragment binds to an epitope comprising an amino acid selected from the group consisting of: V7, L68, E92 and A178.

In one embodiment, the patient sample comprises a tumor tissue sample.

In one embodiment, the tumor is selected from the group consisting of a breast tumor, colon tumor and lung tumor.

In one embodiment, the patient sample is a body fluid.

In one embodiment, the body fluid is selected from the group consisting of blood, lymph, ascetic fluid, gynecological fluid, cystic fluid and urine.

In another aspect, the invention pertains to a method for detecting the presence of a TDGF3 polynucleotide or portion thereof in a sample, the method comprising the steps of:
a) contacting the sample with a nucleic acid molecule which selectively hybridizes to a portion of a transcribed TDGF3 polynucleotide, which portion comprises nucleotides within the TDGF3 coding region encoding an amino acid selected from the group consisting of: V7, L68, E92 and A178;
b) amplifying the transcribed TDGF3 polynucleotide or portion thereof with the nucleic acid molecule by polymerase chain reaction; and
b) detecting amplified TDGF3 polynucleotide, to thereby detect the presence of the TDGF3 polynucleotide or portion thereof in the sample.

In another aspect, the invention pertains to a kit for detecting the presence in a sample of TDGF3 polynucleotide or portion thereof, the kit comprising a nucleic acid molecule that selectively hybridizes with the TDGF3 transcribed polynucleotide.

In another aspect, the invention pertains to a kit for detecting the presence in a sample of TDGF3 polypeptide or portion thereof, the kit comprising an antibody, antibody derivative, or fragment thereof, wherein the antibody or fragment thereof specifically binds with a TDGF3 polypeptide or portion thereof.

In yet another aspect, the invention pertains to a method of assessing whether a cell is transformed, comprising comparing:

a) the level of expression of a TDGF3 gene in a test cell, and

b) the level of expression of a TDGF3 gene in a control non-transformed cell,

wherein a higher level of expression of the TDGF3 gene in the test cell as compared to the level in the control non-transformed cell is an indication that the test cell is transformed.

In one embodiment, the level of expression of the TDGF3 gene in the test cell and in the control cell is assessed by detecting the presence in the test cell and in the control cell of a transcribed polynucleotide or portion thereof, wherein the transcribed polynucleotide comprises the coding region of the TDGF3 gene.

In one embodiment, the transcribed polynucleotide is an mRNA.

In one embodiment, the transcribed polynucleotide is a cDNA.

In one embodiment, the step of detecting further comprises amplifying the transcribed polynucleotide prior to detecting the transcribed polynucleotide.

In one embodiment, the amplifying step comprises a polymerase chain reaction.

In one embodiment, the transcribed polynucleotide is amplified using at least one nucleic acid molecule which selectively hybridizes to the TDGF3 coding region.

In one embodiment, the at least one nucleic acid molecule does not amplify a TDGF1 polynucleotide.

In one embodiment, the at least one nucleic acid molecule hybridizes to a portion of transcribed polynucleotide corresponding to the TDGF3 coding region which spans the nucleotides encoding an amino acid selected from the group consisting of: V7, L68, E92 and A178.

In one embodiment, the at least one nucleic acid molecule comprises a sequence selected from the group consisting of the sequences set forth in Table 2 and Table 3.

In one embodiment, the level of expression of the TDGF3 gene in the test cell and in the control cell is assessed by detecting the presence in the test cell and in the control cell of a protein encoded by the TDGF3 gene using a reagent that specifically binds with the protein.

In one embodiment, the reagent is selected from the group consisting of an antibody, antibody derivative and an antibody fragment.

In one embodiment, the antibody, antibody derivative or antibody fragment binds to the TDGF3 polypeptide and does not bind to a TDGF1 polypeptide.

In one embodiment, the antibody, antibody derivative or antibody fragment binds to an epitope comprised in the extracellular portion of the TDGF3 polypeptide.

In one embodiment, the antibody, antibody derivative or antibody fragment binds to an epitope comprising one or more amino acids selected from the group consisting of: V7, L68, E92 and A178.

In yet another aspect, the invention pertains to a kit for assessing the presence in a sample of transformed cells, the kit comprising an antibody, antibody derivative, or fragment thereof, wherein the antibody or fragment thereof specifically binds with a TDGF3 protein.

In another aspect, the invention pertains to a kit for assessing the presence in a sample of transformed cells, the kit comprising a nucleic acid molecule that selectively hybridizes with a TDGF3 transcribed polynucleotide.

In another aspect, the invention pertains to a method of assessing whether a patient is a suitable candidate for an anti-Cripto antibody therapy, the method comprising comparing:

a) the level of expression of a TDGF3 gene in a patient sample, and

b) the level of expression of a TDGF3 gene in a control non-cancer sample,

wherein a higher level of expression of the TDGF3 gene in the patient sample, as compared to the control non-cancer sample, is an indication that the patient is a suitable candidate for an anti-Cripto antibody therapy.

In another aspect, the level of expression of the TDGF3 gene in the sample is assessed by detecting the presence in the sample of a transcribed polynucleotide or portion thereof, wherein the transcribed polynucleotide comprises the coding region of the TDGF3 gene.

In one embodiment, the transcribed polynucleotide is an mRNA.

In one embodiment, the transcribed polynucleotide is a cDNA.

In one embodiment, the step of detecting further comprises amplifying the transcribed polynucleotide prior to detecting the transcribed polynucleotide.

In one embodiment, the amplifying step comprises a polymerase chain reaction.

In one embodiment, the transcribed polynucleotide is amplified using at least one nucleic acid molecule which selectively hybridizes to the TDGF3 coding region.

In one embodiment, the at least one nucleic acid molecule does not amplify a TDGF1 polynucleotide.

In one embodiment, the at least one nucleic acid molecule hybridizes to a portion of transcribed polynucleotide corresponding to the TDGF3 coding region which spans the nucleotides encoding an amino acid selected from the group consisting of: V7, L68, E92 and A178.

In one embodiment, the at least one nucleic acid molecule comprises a sequence selected from the group consisting of the sequences set forth in Table 2 and Table 3.

In one embodiment, the level of expression of the TDGF3 gene in the sample is assessed by detecting the presence in the sample of a transcribed polynucleotide with a nucleic acid probe which selectively hybridizes with the nucleotide sequence of a transcribed TDGF3 polynucleotide or hybridizes with a portion of a transcribed TDGF3 polynucleotide under stringent hybridization conditions.

In one embodiment, the level of expression of the TDGF3 gene in the sample is assessed by detecting the presence in the sample of a protein encoded by the TDGF3 gene using a reagent that specifically binds with the protein.

In one embodiment, the reagent is selected from the group consisting of an antibody, antibody derivative and an antibody fragment.

In one embodiment, the antibody, antibody derivative or antibody fragment binds to the TDGF3 polypeptide and does not bind to a TDGF1 polypeptide.

In one embodiment, the antibody, antibody derivative or antibody fragment binds to an epitope comprised in the extracellular portion of the TDGF3 polypeptide.

In one embodiment, the antibody, antibody derivative or antibody fragment binds to an epitope comprising one or more amino acids selected from the group consisting of: V7, L68, E92 and A178.

In one embodiment, the patient sample comprises a tumor tissue sample.

In one embodiment, the tumor is selected from the group consisting of a breast tumor, colon tumor and lung tumor.

In one embodiment, the patient sample is a body fluid.

In one embodiment, the body fluid is selected from the group consisting of blood, lymph, ascetic fluid, gynecological fluid, cystic fluid and urine.

In one embodiment, the level of expression of TDGF3 gene in the patient sample differs from the level of expression of the TDGF3 gene in a control non-cancer sample by a factor of at least about 2-fold.

In one embodiment, the level of expression of TDGF3 gene in the patient sample differs from the level of expression of the TDGF3 gene in a control non-cancer sample by a factor of at least about 5-fold.

In one embodiment, the TDGF3 gene is not expressed in the control non-cancer sample.

In another aspect, the invention pertains to a kit for assessing whether a patient is a suitable candidate for an anti-Cripto antibody therapy, the kit comprising an antibody, antibody derivative, or fragment thereof, wherein the antibody or fragment thereof specifically binds with a TDGF3 protein.

In another aspect, the invention pertains to a kit for assessing whether a patient is a suitable candidate for an anti-Cripto antibody therapy, the kit comprising a nucleic acid molecule that selectively hybridizes with a TDGF3 transcribed polynucleotide.

In another aspect, the invention pertains to a method of selecting a composition for inhibiting cellular transformation in a cell, the method comprising:

a) obtaining a sample comprising cells, and

b) separately maintaining aliquots of the sample in the presence of a plurality of test compositions;

c) comparing expression of a TDGF3 gene in each of the aliquots;

d) selecting one of the test compositions which induces a lower level of expression of the TDGF3 gene in the aliquot containing that test composition, relative to other test compositions.

In another aspect, the invention pertains to a method of assessing the carcinogenic potential of a test compound, the method comprising:

a) maintaining separate aliquots of mammalian cells in the presence and absence of the test compound; and

c) comparing expression of a TDGF3 gene in each of the aliquots;

d) wherein a greater level of expression of the TDGF3 gene in the aliquot maintained in the presence of the test compound, relative to the aliquot maintained in the absence of the test compound, is an indication that the test compound possesses carcinogenic potential.

In yet another aspect, the invention pertains to a method of making an isolated monoclonal antibody useful for specifically detecting the presence of a TDGF3 polypeptide or portion thereof in a sample, the method comprising:

isolating a TDGF3 polypeptide or portion thereof,

immunizing a mammal using the isolated polypeptide;

isolating splenocytes from the immunized mammal;

fusing the isolated splenocytes with an immortalized cell line to form hybridomas; and

screening individual hybridomas for production of an antibody which specifically binds with the TDGF3 polypeptide; and

isolating the antibody produced by the hybridoma, to thereby isolate a monoclonal antibody useful for specifically detecting the presence of a TDGF3 polypeptide or portion thereof in a sample.

In another aspect, the invention pertains to a monoclonal antibody produced using a method of the invention.

In one aspect, the invention pertains to a method for detecting the presence of a TDGF1 polynucleotide or portion thereof in a sample, the method comprising the steps of:
a) contacting the sample with a nucleic acid molecule which selectively hybridizes to a transcribed TDGF1 polynucleotide, wherein the transcribed TDGF3 polynucleotide comprises the coding region of the TDGF1 gene; and
b) determining whether the nucleic acid molecule binds to the polynucleotide in the sample, to thereby detect the presence of the TDGF1 polynucleotide or portion thereof in the sample.

In one embodiment, the transcribed TDGF1 polynucleotide is mRNA.

In one embodiment, the transcribed TDGF1 polynucleotide is cDNA.

In one embodiment, the method comprises the step of amplifying the transcribed TDGF1 polynucleotide with the nucleic acid molecule.

In one embodiment, the amplification step comprises a polymerase chain reaction.

In one embodiment, binding of the nucleic acid molecule to the transcribed TDGF1 polynucleotide is determined by detecting amplified TDGF1 polynucleotide.

In one embodiment, the transcribed polynucleotide is amplified using at least one nucleic acid molecule which selectively hybridizes to the transcribed TDGF1 polynucleotide.

In one embodiment, the at least one nucleic acid molecule does not amplify a TDGF3 polynucleotide.

In one embodiment, the at least one nucleic acid molecule hybridizes to a portion of the transcribed TDGF1 polynucleotide, which portion comprises nucleotides within the TDGF3 coding region encoding an amino acid selected from the group consisting of: A7, P68, G92, V178, V22 and Y43.

In one embodiment, the at least one nucleic acid molecule comprises a sequence selected from the group consisting of the sequences set forth in Table 2 and Table 3.

In one aspect, the invention pertains to a method for detecting the presence of a TDGF1 polypeptide or portion thereof in a sample, the method comprising the steps of:
a) contacting the sample with a reagent which selectively binds to a TDGF3 polypeptide; and
b) determining whether the reagent binds to the polypeptide in the sample, to thereby detect the presence of the TDGF3 polypeptide or portion thereof in the sample.

In one embodiment, the reagent is selected from the group consisting of an antibody, antibody derivative and an antibody fragment.

In one embodiment, the antibody, antibody derivative or antibody fragment binds to the TDGF3 polypeptide and does not bind to a TDGF1 polypeptide.

In one embodiment, the antibody, antibody derivative or antibody fragment binds to an epitope comprised in the extracellular portion of the TDGF3 polypeptide.

In one embodiment, the antibody, antibody derivative or antibody fragment binds to an epitope comprising an amino acid selected from the group consisting of: V7, L68, E92 and A178.

In one embodiment, the patient sample comprises a tumor tissue sample.

In one embodiment, the tumor is selected from the group consisting of a breast tumor, colon tumor and lung tumor.

In one embodiment, the patient sample is a body fluid.

In one embodiment, the body fluid is selected from the group consisting of blood, lymph, ascetic fluid, gynecological fluid, cystic fluid and urine.

In one aspect, the invention pertains to a method for detecting the presence of a TDGF1 polynucleotide or portion thereof in a sample, the method comprising the steps of:
a) contacting the sample with a nucleic acid molecule which selectively hybridizes to a portion of a transcribed TDGF1 polynucleotide, which portion comprises nucleotides within the TDGF1 coding region encoding an amino acid selected from the group consisting of: A7, P68, G92, V178, V22 and Y43;
b) amplifying the transcribed TDGF1 polynucleotide or portion thereof with the nucleic acid molecule by polymerase chain reaction; and
b) detecting amplified TDGF1 polynucleotide, to thereby detect the presence of the TDGF1 polynucleotide or portion thereof in the sample.

In one aspect, the invention pertains to a kit for detecting the presence in a sample of TDGF1 polynucleotide or portion thereof, the kit comprising a nucleic acid molecule that selectively hybridizes with the TDGF1 transcribed polynucleotide.

In one aspect, the invention pertains to a kit for detecting the presence in a sample of TDGF1 polypeptide or portion thereof, the kit comprising an antibody, antibody derivative, or fragment thereof, wherein the antibody or fragment thereof specifically binds with a TDGF1 polypeptide or portion thereof.

In another aspect, the invention pertains to an isolated nucleic acid molecule for specifically detecting a TDGF1 polynucleotide, wherein the nucleic acid molecule is selected from the group of sequences set forth in Table 2 and Table 3.

In another aspect, the invention pertains to an isolated nucleic acid molecule for specifically detecting a TDGF3 polynucleotide, wherein the nucleic acid molecule is selected from the group of sequences set forth in Table 2 and Table 3.

### BRIEF DESCRIPTION OF THE DRAWINGS

FIGS. 1A-C depict the nucleotide sequences and polypeptide sequences encoding Cripto-1 and Cripto-3. (A) Peptide sequence alignment of proteins encoded by TDGF1 and TDGF3. The top lines are Cripto 1 sequence (SEQ ID NO:1) and the bottom lines are Cripto 3 sequence (SEQ ID NO:2). The common sequence is in the middle. The positions with different amino acid residues between the two proteins are indicated by dots. The variable amino acid sites in Cripto 1 caused by a SNP in TDGF1 are indicated with asterisks, and the sites with fixed amino acid differences between the two proteins are boxed. The signal peptide is in bold face. The potential fucosylation site is underlined. (B) Nucleic acid sequence encoding Cripto-1 (SEQ ID NO:3). (C) Nucleic acid sequence encoding Cripto-3 (SEQ ID NO:4).

FIGS. 2A-2B depict the results of PCR amplification of a TDGF cDNA fragment. (A) cDNA purity test. The position of oligos relative to TDGF1 genes and the expected outcome from PCRs with cDNA and genomic DNAs are indicated. Lanes 14: cDNA from 4 breast tumors; lanes 5-8: cDNA from 4 colon tumors; lanes 9-12: cDNA from 4 lung tumors; lane 13: 100 bp DNA marker; lanes 14-25: genomic DNA from same set of tissue samples as in lane 1-12. (B) Inter-exon PCRs. The position of oligos relative to TDGF genes and transcripts, as well as the expected outcome from PCRs with cDNA and genomic DNAs are indicated. The 1374 bp DNA fragment is from the TDGF1 gene, the 286 bp DNA fragment amplified from genomic DNA is from the TDGF3 gene, and the 286 bp DNA fragment amplified from cDNAs is from the TDGF1 and/or TDGF3 cDNA, since the cDNA templates are free of genomic DNA contamination.

FIGS. 3A-3B depict the results from transcript-specific PCRs. (A) Results of PCR with TDGF1 transcript-specific oligo pairs. (B) Results of PCR with TDGF3 specific oligo pairs. Lane 1: Normal breast #1; Lane 2: Normal breast #3; Lane 3: Normal breast #4; Lane 4: Normal lung #; Lane 5: Normal lung #4; Lanes 6-12: Breast cancers; Lane 13; Normal colon; Lanes 14-15: Colon cancers; Lanes 16-18; Normal lung; Lane 19: Normal matched lung; Lanes 20-21: Lung cancers; Lane 22: gDNA control; Lane 23: 100 bp DNA markers.

FIGS. 4A-4B depict the results from a FACS analysis of Cripto 1 and Cripto 3 on the cell surface. (A) FACS analysis of TDGF1 and TDGF3 positive cell lines using an antibody against Cripto. (a) NCCIT cells (TDGF1 positive); (b) BT474 cells (TDGF3 positive); (c) and (d) negative controls with anti-mouse IgG. The results show that both Cripto 1 and Cripto 3 proteins are present on the cell surface and can be detected by an anti-Cripto antibody. (B) FACS analysis of cells transfected with TDGF1 or TDGF3. (a) T47D cells transfected with the TDGF1 gene; (b) T47D cells transfected with the TDGF3 gene; (c) and (d) negative controls with anti-mouse IgG. The results show that Cripto 1 and Cripto 3 encoded by the transfected genes TDGF1 and TDGF3 are expressed on the cell surface and are recognized by an anti-Cripto antibody.

FIG. 5 depicts results showing that Cripto-1 and Cripto-3 can signal through Nodal in F9 cells. F9 cripto−/− cells were transfected with plasmids expressing (n2)₇-luciferase, FAST and either no addition (column 1), or the addition of human Cripto-1 (column 2) or human Cripto-3 (column 3).

### DETAILED DESCRIPTION OF THE INVENTION

There are at least seven CRIPTO genes and pseudogenes in the human genome, named as TDGF1 through TDGF7. TDGF1 is located on Chromosome 3 p23-21 region and, prior to the instant invention, was widely believed to be the only structural gene for Cripto protein (Table 1).

TABLE 1
TDGF gene list

| | | Genomic | | |
|---|---|---|---|---|
| TDGF genes | Classification | location | ORF | Expression |
| TDGF1 | Structural | Chr 3p23-p21 | Intact | Yes |
| TDGF2 | Pseudogene | Chr 2q37 | Broken | |
| TDGF3 | Pseudogene | Chr Xq21-q22 | Intact | Unknown |
| TDGF4 | Pseudogene | Chr 6p25 | Broken | |
| TDGF5 | Pseudogene | Chr 8 | broken | |
| TDGF6 | Pseudogene | Chr 3q22 | broken | |
| TDGF7 | Pseudogene | Chr 19q13 | broken | |

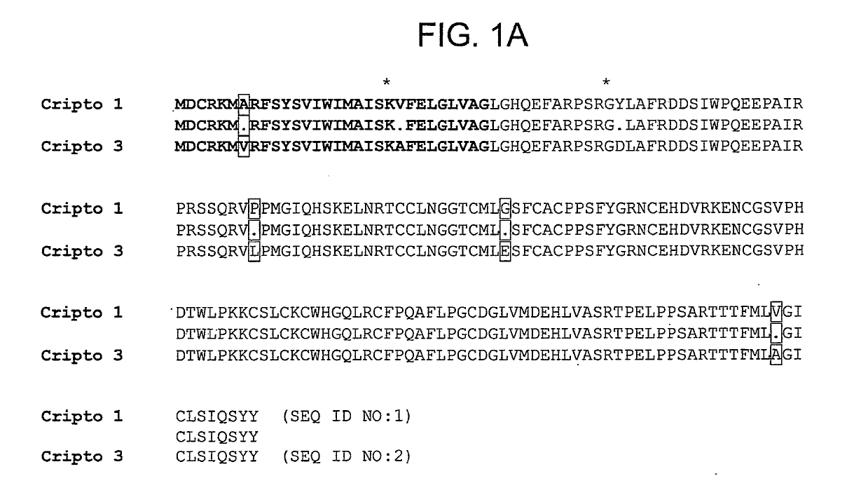

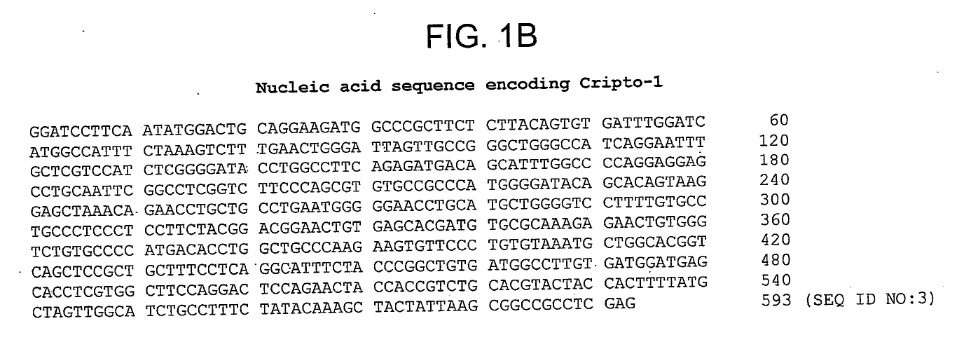

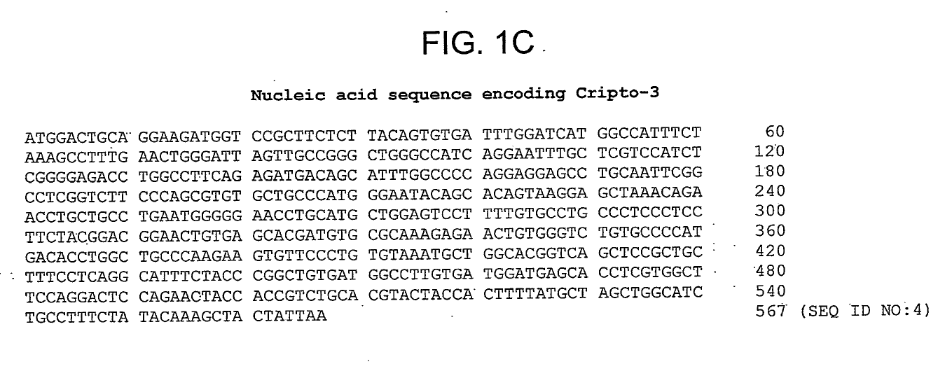

Among the 6 presumed pseudogenes, TDGF3 on the X chromosome (Xq28) has an intact open reading frame that could encode a predicted protein (Cripto-3) having six different amino acids as compared to the published Cripto-1 protein reference sequence (Scognamiglio B 1999) (FIG. 1A; SEQ ID NO: 1). This gene is intronless and appears to be derived from an insertion of the TDGF1 cDNA into the human genome during evolution.

The present invention is based, at least in part, on the surprising discovery that a large number of Cripto cDNA isolates from multiple cancer tissue and cell lines were derived from the TDGF3 (Cripto-3) gene rather than the TDGF1 (Cripto-1) gene. Both the TDGF1 and TDGF3 genes were transcribed and translated in a number of human normal and cancer tissues. When Cripto-expressing tissue samples were examined, both genes were expressed at similar levels in only a small number of cases, while in most cases either TDGF1 or TDGF3 was predominately expressed. In particular, the present invention is based on the discovery that while TDGF1 may be expressed in some normal tissues, TDDGF3, rather than TDGF1, is overexpressed on most Cripto-expressing tumors.

Accordingly, compositions, kits and methods are provided herein for detecting the presence of a marker of the invention, e.g., Cripto-3 and/or Cripto-1, in a sample, e.g., by specifically detecting the expression of a marker, e.g., Cripto-3 and/or Cripto-1, polynucleotide or polypeptide, in a sample. These compositions, kits and methods are useful for determining the phenotype of a tumor, e.g., whether a tumor is a Cripto-1 or Cripto-3 expressing tumor. Methods are also provided herein for assessing whether a cell is transformed. These methods involve comparing the level of expression of a TDGF3 gene in the cell to the level of expression of the TDGF3 gene in a control, non-transformed cell. Methods are further provided herein for assessing whether a patient is a suitable candidate for an anti-Cripto antibody therapy. These methods involve comparing the level of expression of a TDGF3 gene in a patient sample to the level of expression of the TDGF3 gene in a control, non-cancer sample.

Various aspects of the invention are described in further detail in the following subsections:

### I. DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

As used herein, the term “marker” includes markers, e.g., Cripto-3 (TDGF3) and/or Cripto-1 (TDGF1), which are believed to be involved in the transformation of a cell or the development (including maintenance, progression, angiogenesis, and/or metastasis) of a proliferative disease, e.g., cancer. A “marker” includes markers, e.g., Cripto-3 and/or Cripto-1, which are useful in the assessment of whether a cell is transformed. A “marker” also includes markers, e.g., Cripto-3 and/or Cripto-1, which are useful in the assessment of whether a patient is a suitable candidate for an anti-Cripto antibody therapy. The terms “TDGF3” and “Cripto-3” are used interchangeably herein. The terms “TDGF I” and “Cripto-1” are used interchangeably herein.

A marker of the invention may also be useful in the diagnosis of a proliferative disease (e.g., cancer), e.g., over- or under-activity, emergence, expression, growth, remission, recurrence or resistance of a proliferative disease before, during or after therapy. A marker of the invention may further be useful for the diagnosis of tumor grade, tumor prognosis, and treatment response of a tumor. The predictive functions of the marker may be confirmed by, e.g., (1) overexpression or underexpression (e.g., by ISK, Northern Blot, or qPCR), increased or decreased protein level (e.g., by IHC), or increased or decreased activity (determined by, for example, modulation of a pathway in which the marker is involved), in a human proliferative disease, e.g., cancer (e.g., in more than about 2%, 3%, 5%, 5%, 6%, 7%, 8%, 9%, 10%, 11%, 12%, 13%, 14%, 15%, 20%, 25%, 50% or more of a human proliferative disease, e.g., a cancer); (2) its presence or absence in a biological sample, e.g., a sample comprising tissue, cells or a biological fluid from a subject (e.g. a human subject) afflicted with a proliferative disease, e.g., cancer; or (3) its presence or absence in clinical subset of patients with a proliferative disease, e.g., cancer (e.g., those responding to a particular therapy or those developing resistance).

A “marker nucleic acid” is a nucleic acid (e.g., DNA, mRNA, cDNA) encoded by or corresponding to a marker of the invention, e.g., Cripto-3 and/or Cripto-1. For example, such marker nucleic acid molecules include DNA (e.g., genomic DNA or cDNA) comprising the entire or a partial sequence of the nucleic acid sequence of the Cripto-3 gene or the complement or hybridizing fragment of such a sequence. The marker nucleic acid molecules also include RNA, e.g., mRNA, comprising the entire or a partial sequence of the nucleic acid sequence of the Cripto-3 gene or the complement of such a sequence, wherein all thymidine residues are replaced with uridine residues.

A “marker protein” or “marker polypeptide” is a protein encoded by or corresponding to a marker of the invention, e.g., Cripto-3 and/or Cripto-1. A marker protein comprises the entire amino acid sequence of a protein encoded by the polynucleotide sequence of the Cripto-3 gene. The terms “protein” and “polypeptide” are used interchangeably herein.

The term “altered amount” or “modulated amount”, used interchangeably herein, of a marker, or “altered level” or “modulated level”, used interchangeably herein, of a marker refers to a modulated, e.g., increased or decreased, expression level of a marker gene in a sample, e.g., a sample from a subject afflicted with a proliferative disease (e.g., cancer), as compared to the expression level of the marker in a control sample (e.g., sample from normal, non-cancerous tissue, e.g., adjacent normal tissue, or sample from a healthy subject not afflicted with a proliferative disease, e.g., cancer). The term “altered amount” or “modulated amount” of a marker also includes a modulated, e.g., an increased or decreased, protein level of a marker in a sample, e.g., a sample from a subject afflicted with a proliferative disease (e.g., cancer), as compared to the protein level of the marker in a normal, control sample.

The term “altered level of expression” used interchangeably herein with “modulated level of expression” of a marker refers to an expression level of a marker in a sample e.g., a sample derived from a patient suffering from a proliferative disease (e.g., cancer), that is modulated, e.g., is greater or less, than the expression level of the marker in a control sample (e.g., sample from normal, non-cancerous tissue, e.g., adjacent normal tissue, or sample from a healthy subject not afflicted with a proliferative disease, e.g., cancer) by a statistically significant amount, e.g., by an amount that is greater than the standard error of the assay employed to assess expression. Preferably, the expression level of the marker in the test sample is modulated, e.g., is greater or less, than the expression level of the marker in a control sample by at least two, and more preferably three, four, five or ten or more fold and preferably, the average expression level of the marker in several control samples.

An “overexpression” or “higher level of expression” or “greater level of expression” of a marker refers to an expression level in a sample that is greater than the expression level of the marker in a control sample (e.g., sample from normal, non-cancerous tissue, e.g., adjacent normal tissue, or sample from a healthy subject not afflicted with a proliferative disease, e.g., cancer) by a statistically significant amount, e.g., by an amount greater the standard error of the assay employed to assess expression, and is preferably at least twice, and more preferably three, four, five or ten or more times the expression level of the marker in a control sample and, preferably, the average expression level of the marker in several control samples.

An “underexpression” or “lower level of expression” of a marker refers to an expression level in a sample that is less than the expression level of the marker in a control sample (e.g., sample from normal, non-cancerous tissue, e.g., adjacent normal tissue, or sample from a healthy subject not afflicted with a proliferative disease, e.g., cancer) by a statistically significant amount, e.g., by an amount greater than the standard error of the assay employed to assess expression, and is preferably at least twice, and more preferably three, four, five or ten or more times less than the expression level of the marker in a control sample and, preferably, the average expression level of the marker in several control samples.

The amount of a marker, e.g., expression of a marker, or protein level of a marker, in a sample is “significantly” higher or lower than the amount of a marker in a control sample (e.g., sample from normal, non-cancerous tissue, e.g., adjacent normal tissue, or sample from a healthy subject not afflicted with a proliferative disease, e.g., cancer), if the amount of the marker is greater or less, respectively, than the level in the control sample by an amount greater than the standard error of the assay employed to assess amount, and preferably at least twice, and more preferably three, four, five, ten or more times that amount. Alternately, the amount of the marker in the sample can be considered “significantly” higher or lower than the amount in a control sample if the amount is at least about two, and preferably at least about three, four, or five times, higher or lower, respectively, than the amount of the marker in the control sample.

The term “altered activity” used interchangeably herein with “modulated activity” of a marker, e.g., Cripto-3 and/or Cripto-1, refers to an activity of a marker which is modulated, e.g., increased or decreased, in a disease state, e.g., in a proliferative disease (e.g., cancer), as compared to the activity of the marker in a normal, control sample. Altered or modulated activity of a marker may be the result of, for example, altered or modulated expression of the marker, altered or modulated protein level of the marker, altered or modulated structure of the marker, or, e.g., an altered or modulated interaction with other proteins involved in the same or different pathway as the marker, or altered or modulated interaction with transcriptional activators or inhibitors, or altered methylation status.

The term “altered structure” used interchangeably herein with “modulated structure” of a marker, e.g., Cripto-3 and/or Cripto-1, refers to the presence of mutations or allelic variants within the marker gene or maker protein, e.g., mutations which affect expression or activity of the marker, as compared to the normal or wild-type gene or protein. For example, mutations include, but are not limited to, substitutions, deletions, or addition mutations. Mutations may be present in the coding or non-coding region of the marker.

A “transcribed polynucleotide” is a polynucleotide (e.g. an RNA, a cDNA, or an analog of one of an RNA or cDNA) which is complementary to or homologous with all or a portion of a mature RNA made by transcription of a marker of the invention, e.g., Cripto-3, and normal post-transcriptional processing (e.g. splicing), if any, of the transcript, and reverse transcription of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid molecule is complementary to a second region of the same or a different nucleic acid molecule if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, more preferably, 60%, 70%, 80%, 90%, 95% or more.

The term “probe” refers to any molecule which is capable of selectively binding to a specifically intended target molecule, for example a marker of the invention, e.g., Cripto-3 and/or Cripto-1. Probes can be either synthesized by one skilled in the art, or derived from appropriate biological preparations. For purposes of detection of the target molecule, probes may be, specifically designed to be labeled, as described herein. Examples of molecules that can be utilized as probes include, but are not limited to, RNA, DNA, proteins, antibodies, and organic monomers.

A “nucleic acid probe” or “primer” refers to any nucleic acid molecule which is capable of selectively hybridizing to a marker polynucleotide of the invention, e.g., a Cripto-3 polynucleotide and/or Cripto-1 polynucleotide. A “nucleic acid probe” or “primer” includes any nucleic acid molecule which is capable of selectively hybridizing to a Cripto-3 polynucleotide, e.g., a Cripto-3 transcribed polynucleotide, such that a Cripto-3 polynucleotide is selectively detected, e.g., such that a Cripto-1 polynucleotide, e.g., Cripto-1 transcribed polynucleotide, is less efficiently detected or is not detected. A “nucleic acid probe” or “primer” also includes any nucleic acid molecule which is capable of selectively hybridizing to a Cripto-1 polynucleotide, e.g., a Cripto-1 transcribed polynucleotide, such that a Cripto-1 polynucleotide is selectively detected, e.g., such that a Cripto-3 polynucleotide, e.g., Cripto-3 transcribed polynucleotide, is less efficiently detected or is not detected.

A marker or probe (e.g., nucleic acid probe or primer) is “fixed” to a substrate if it is covalently or non-covalently associated with the substrate such the substrate can be rinsed with a fluid (e.g. standard saline citrate, pH 7.4) without a substantial fraction of the marker dissociating from the substrate.

As used herein, a “naturally-occurring” nucleic acid molecule refers to an RNA or DNA molecule having a nucleotide sequence that occurs in nature (e.g. encodes a natural protein).

As used herein, a “proliferative disease” refers to any disease associated with undesired cell proliferation, e.g., cancer. Non-limiting examples of a proliferative disease, as used herein, include breast cancer, lung cancer, colorectal cancer, testicular cancer, ovarian cancer, renal cancer, uterine cancer, cervical cancer, prostate cancer, bladder cancer, pancreatic cancer, stomach cancer, central nervous system cancer, mealanoma, lymphoma and leukemia.

A proliferative disease, e.g., cancer, is “modulated”, e.g., “inhibited” if at least one symptom of the proliferative disease is alleviated, terminated, slowed, or prevented. As used herein, a proliferative disease, e.g., cancer, is also “inhibited” if relapse, recurrence or metastasis of the proliferative disease (e.g., tumor) is reduced, slowed, delayed, or prevented.

As used herein, a “subject” refers to vertebrates, particularly members of a mammalian species, and includes but is not limited to domestic animals, sports animals, and primates, including humans.

As used herein, the term “promoter”, “regulatory sequence”, or “promotor element” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a spatially or temporally restricted manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell

A “tissue-specific promoter”, “spatially-restricted promoter or regulatory sequence”, or “spatially restricted promotor element” is a nucleotide sequence which, when operably linked with a polynucleotide which encodes or specifies a gene product, causes the gene product to be produced in a cell substantially only if the cell is a cell of the tissue type corresponding to the promoter.

A “temporally-restricted promoter or regulatory sequence” or “temporally restricted promotor element” is a nucleotide sequence which, when operably linked with a polynucleotide which encodes or specifies a gene product, causes the gene product to be produced in a living human cell substantially only if the cell is at a particular developmental stage or is subjected to an agent which induces the expression of the promoter, e.g., tetracycline or tamoxifen.

A kit is any manufacture (e.g. a package or container) comprising at least one reagent, e.g. a probe, for specifically detecting a marker of the invention, the manufacture being promoted, distributed, or sold as a unit for performing the methods of the present invention.

### II. USES OF THE INVENTION

The present invention is based, in part, on the identification of markers, e.g., Cripto-3 and/or Cripto-1, involved in cell transformation, e.g., a marker preferentially expressed in cells afflicted with a proliferative disease, e.g., cancer, as compared to control (i.e., non-afflicted or normal) cells. The markers of the invention may be DNA, cDNA, RNA, or polypeptide molecules which can be detected in one or both of normal and afflicted cells.

The amount, structure, and/or activity, e.g., the presence, absence, expression level, protein level, protein activity, presence of mutations, e.g., mutations which affect activity of the marker (e.g., substitution, deletion, or addition mutations), and/or methylation status, of the marker in a sample, e.g., a sample containing tissue or cells, e.g., tumor tissue or cells, or a sample containing biological fluid, e.g., whole blood, serum, plasma, buccal scrape, saliva, spinal fluid, cerebrospinal fluid, urine, stool, is herein correlated with the transformation state of the cells.

The invention thus provides compositions, kits and methods for detecting the presence of a marker, e.g., Cripto-3 and/or Cripto-1, in a sample. These compositions, kits and methods are useful for determining the phenotype, e.g., Cripto-expressing phenotype, of a tumor, e.g., whether the tumor expresses Cripto-1 or Cripto-3. These compositions, kits, and methods are further useful for assessing the transformation state of cells as well as for assessing whether a patient is a suitable candidate for an anti-Cripto antibody therapy.

The compositions, kits, and methods of the invention have the following uses, among others:
1) detecting a marker, e.g., Cripto-1 and/or Cripto-3, e.g., a Cripto-3 polynucleotide or polypeptide, in a sample;
2) assessing whether a cell is transformed or at risk for becoming transformed;
3) assessing the presence of transformed or malignant cells in a sample;
4) assessing the benign or malignant nature of a tumor in a subject;
5) assessing the phenotype of a tumor, e.g., whether the tumor is a Cripto-1 or Cripto-3 expressing tumor;
6) assessing whether a subject is afflicted with a proliferative disease, disorder or condition;
7) assessing whether a subject afflicted with a tumor is a suitable candidate for an anti-Cripto antibody-based treatment;
8) predicting responsiveness of a subject afflicted with a tumor to treatment, e.g., an anti-Cripto antibody-based treatment;
9) making antibodies, antibody fragments or antibody derivatives that are useful for detecting a Cripto-3 polypeptide, assessing whether a cell is transformed, assessing whether a patient is a suitable candidate for an anti-Cripto antibody therapy, treating a proliferative disease, disorder or condition and/or assessing whether a subject is afflicted with a proliferative disease, disorder or condition;
10) assessing the efficacy of one or more test compounds for inhibiting transformation of a cell;
11) assessing the carcinogenic potential of a test compound.

The invention thus includes compositions, kits and methods for detecting the presence of a marker polynucleotide, e.g., a Cripto-3 polynucleotide or Cripto-1 polynucleotide, in a sample. In one embodiment, the methods comprise contacting the sample with a nucleic acid molecule which selectively hybridizes to a transcribed Cripto-3 polynucleotide, wherein the transcribed Cripto-3 polynucleotide comprises the coding region of the Cripto-3 gene, and determining whether the nucleic acid molecule binds to the polynucleotide in the sample. In another embodiment, the methods comprise contacting the sample with a nucleic acid molecule which selectively hybridizes to a transcribed Cripto-1 polynucleotide, wherein the transcribed Cripto-1 polynucleotide comprises the coding region of the Cripto-1 gene, and determining whether the nucleic acid molecule binds to the polynucleotide in the sample.

The invention further includes compositions, kits and methods for detecting the presence of a marker polypeptide, e.g., a Cripto-3 polypeptide or Cripto-1 polypeptide, in a sample. In one embodiment, the methods comprise contacting the sample with a reagent which selectively binds to a Cripto-3 polypeptide, and determining whether the reagent binds to the polypeptide in the sample. In another embodiment, the methods comprise contacting the sample with a reagent which selectively binds to a Cripto-1 polypeptide, and determining whether the reagent binds to the polypeptide in the sample.

These compositions, kits and methods for detecting the presence of a marker of the invention in a sample are useful for determining the phenotype of a tumor, e.g., whether a tumor is a Cripto-3 or Cripto-1 expressing tumor. These methods are further useful for assessing whether a cell is transformed, e.g., for diagnosing cancer. These compositions, kits and methods are further useful for assessing whether a patient is a suitable candidate for an anti-Cripto antibody-based therapy.

Accordingly, the invention provides a method of assessing whether a cell is transformed. This method comprises comparing the amount, structure or activity, e.g., the presence, absence, expression level, protein level, protein activity, presence of mutations, e.g., mutations which affect activity of the marker (e.g., substitution, deletion, or addition mutations), and/or methylation status, of a marker, e.g., Cripto-3, in a test cell with the level in a normal, non-transformed cell. A significant difference, e.g., increase, in the amount, structure, or activity of the marker in the test cell as compared to the normal non-transformed cell is an indication that the cell is transformed.

The invention further provides a method of determining the Cripto-expressing phenotype of a tumor. This method comprises comparing the amount, structure or activity of Cripto-3 in a tumor sample with the amount, structure or activity of Cripto-1 in the tumor sample. A significant difference, e.g., increase, in the amount, structure, or activity of Cripto-3 as compared to Cripto-1 in the tumor sample is an indication that the tumor is a Cripto-3 expressing tumor. A significant difference, e.g., increase, in the amount, structure, or activity of Cripto-1 as compared to Cripto-3 in the tumor sample is an indication that the tumor is a Cripto-1 expressing tumor.

The invention further provides a method of assessing whether a patient is a suitable candidate for an anti-Cripto antibody therapy. This method comprises comparing the amount, structure, and/or activity, e.g., the presence, absence, copy number, expression level, protein level, protein activity, presence of mutations, e.g., mutations which affect activity of the marker (e.g., substitution, deletion, or addition mutations), and/or methylation status, of a marker, e.g., Cripto-3, in a patient sample with the level in a control, non-cancer sample. A significant difference, e.g., increase, in the amount, structure, or activity of the marker, e.g., Cripto-3, in the patient sample as compared to the level in the control non-cancer sample is an indication that the patient is a suitable candidate for an anti-Cripto antibody therapy.

In addition, as a greater number of subject samples are assessed for altered amount, structure, and/or activity of the marker, e.g., Cripto-3 and/or Cripto-1, and the outcomes of the individual subjects from whom the samples were obtained are correlated, it will also be confirmed that an altered amount, structure, and/or activity of the marker is strongly correlated with a particular type of cancer or tumor, or with a cancer or tumor having a particular response to a therapy, e.g., an anti-Cripto antibody therapy, e.g., a positive or negative response to an anti-Cripto antibody therapy. The compositions, kits, and methods of the invention are thus useful for characterizing one or more of the stage, grade, histological type, benign/premalignant/malignant nature of, and predicted response to or outcome of an anti-Cripto antibody therapy of, e.g., a cancer or tumor, in a subject.

It is recognized that the compositions, kits, and methods of the invention will be of particular utility to subjects having an enhanced risk of developing a proliferative disease, disorder or condition, and their medical advisors. Subjects recognized as having an enhanced risk of developing a proliferative disease, disorder or condition, include, for example, subjects having a familial history of a proliferative disease, disorder or condition, subjects identified as having a mutant oncogene (i.e. at least one allele), and subjects of advancing age.

A modulation, e.g., an alteration, of e.g. amount, structure, and/or activity of a marker, e.g., Cripto-3 and/or Cripto-1, in normal (i.e. non-afflicted) human tissue can be assessed in a variety of ways. In one embodiment, the normal level of expression is assessed by assessing the level of expression of the marker in a portion of cells which appear to be non-afflicted and by comparing this normal level of expression with the level of expression in a portion of the cells which are suspected of being diseased or afflicted. Alternately, and particularly as further information becomes available as a result of routine performance of the methods described herein, population-average values for “normal” amount, structure, and/or activity of the markers of the invention may be used. In other embodiments, the “normal” amount, structure, and/or activity of a marker may be determined by assessing the amount, structure, and/or activity of the marker in a subject sample obtained from a non-proliferative disease-, disorder- or condition-afflicted subject, from a subject sample obtained from a subject before the suspected onset of a proliferative disease, disorder, or condition in the subject, from archived subject samples, and the like.

The invention includes compositions, kits, and methods for detecting the presence of a marker of the invention, e.g, Cripto-3 and/or Cripto-1, in a sample (e.g. an archived tissue sample or a sample obtained from a subject). The invention further includes compositions, kits and methods for determining the Cripto-expressing phenotype of a tumor, e.g., whether the tumor is a Cripto-1 or Cripto-3 expressing tumor. The invention further includes compositions, kits, and methods for assessing whether a cell is transformed. The invention further includes compositions, kits and methods for assessing whether a patient is a suitable candidate for an anti-Cripto antibody therapy. Where necessary, the compositions, kits, and methods are adapted for use with certain types of samples. For example, when the sample is a parafinized, archived human tissue sample, it may be necessary to adjust the ratio of compounds in the compositions of the invention, in the kits of the invention, or the methods used. Such methods are well known in the art and within the skill of the ordinary artisan.

The invention thus includes a kit for detecting or assessing the amount, e.g., expression, of a marker of the invention, e.g., Cripto-3 and/or Cripto-1, in a sample (e.g., a tissue sample from a subject). The invention further includes a kit for assessing the presence of transformed cells (e.g. in a sample such as a subject sample). The invention further includes a kit for assessing whether a patient is a suitable candidate for an anti-Cripto antibody therapy. The kit may comprise one or more reagents capable of identifying a marker of the invention, e.g., binding specifically with a nucleic acid or polypeptide corresponding to a marker of the invention. Suitable reagents for binding with a polypeptide corresponding to a marker of the invention include antibodies, antibody derivatives, antibody fragments, and the like. Suitable reagents for binding with a nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kits of the invention may optionally comprise additional components useful for performing the methods of the invention. By way of example, the kit may comprise fluids (e.g., SSC buffer) suitable for annealing complementary nucleic acids or for binding an antibody with a protein with which it specifically binds, one or more sample compartments, an instructional material which describes performance of a method of the invention, a sample of normal cells, a sample of neuroglial cells, and the like.

A kit of the invention may comprise a reagent useful for determining protein level or protein activity of a marker.

The invention also includes a method of making an isolated monoclonal antibody useful in methods and kits of the present invention. Monoclonal antibodies may be made using methods known to those of skill in the art. For example, a protein corresponding to a marker of the invention or immunogenic portion thereof, e.g., Cripto-3, may be isolated (e.g., by purification from a cell in which it is expressed or by transcription and translation of a nucleic acid encoding the protein in vivo or in vitro using known methods) and a vertebrate, preferably a mammal such as a mouse, rat, rabbit, or sheep, is immunized using the isolated protein. The vertebrate may optionally (and preferably) be immunized at least one additional time with the isolated protein, so that the vertebrate exhibits a robust immune response to the protein. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein. The invention also includes hybridomas made by this method and antibodies made using such hybridomas. Other methods of making antibodies are known in the art and are described in more detail infra.

The invention also includes a method of assessing the efficacy of a test compound for modulating, e.g., inhibiting, transformation of a cell. As described above, differences in the amount of the markers of the invention, or level of expression of the invention, correlate with the transformed state of cells. Changes in the levels of amount, e.g., expression, of the markers of the invention may result from the transformed state of cells, or may induce, maintain, and promote the transformed state. Thus, compounds which modulate, e.g., inhibit, a proliferative disease, disorder, or condition, in a subject may cause a change, e.g., a change in expression of a marker of the invention to a level nearer the normal level for that marker (e.g., the amount, e.g., expression for the marker in non-afflicted cells).

This method thus comprises comparing the amount, e.g., expression of a marker in a first cell sample maintained in the presence of the test compound and the amount, e.g., expression of the marker in a second cell sample maintained in the absence of the test compound. A significant modulation, e.g., decrease, in the amount, e.g. expression, of a marker is an indication that the test compound modulates transformation of a cell. The cell samples may, for example, be aliquots of a single sample of normal cells obtained from a subject, pooled samples of normal cells obtained from a subject, cells of a normal cell lines, aliquots of a single sample of afflicted cells obtained from a subject, pooled samples of afflicted cells obtained from a subject, cells from an animal model of a proliferative disease, disorder, or condition, or the like

As described above, the transformed state of a cell is correlated with changes in the amount of the marker, e.g., Cripto-3, of the invention. Thus, compounds which induce increased expression or activity of the marker can induce cell carcinogenesis or a proliferative disease, disorder or condition. The invention also includes a method for assessing the human cell carcinogenic potential of a test compound. This method comprises maintaining separate aliquots of human cells in the presence and absence of the test compound. Expression of a marker, e.g., Cripto-3, in each of the aliquots is compared. A significant modulation, e.g., a significant increase, in the amount of a marker in the aliquot maintained in the presence of the test compound (relative to the aliquot maintained in the absence of the test compound) is an indication that the test compound possesses human cell carcinogenic potential or the ability to induce a proliferative disease, disorder or condition. The relative disease causing potential of various test compounds can be assessed by comparing the degree of enhancement of the amount of the marker.

### III. ISOLATED NUCLEIC ACID MOLECULES

One aspect of the invention pertains to nucleic acid molecules that correspond to a marker of the invention, e.g., Cripto-3 and/or Cripto-1, including nucleic acids which encode a marker polypeptide or a portion of such a polypeptide. Nucleic acid molecules of the invention also include nucleic acid molecules sufficient for use as hybridization probes to identify nucleic acid molecules that correspond to a marker gene, e.g., Cripto-3 and/or Cripto-1, including nucleic acid molecules which encode a marker polypeptide, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA) and RNA molecules (e.g., mRNA) and analogs of the DNA or RNA generated using nucleotide analogs. The nucleic acid molecule can be single-stranded or double-stranded.

In one embodiment, a nucleic acid molecule of the invention is an isolated nucleic acid molecule. An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A nucleic acid molecule of the present invention, e.g., a nucleic acid molecule encoding a Cripto-3 protein or fragment thereof or a Cripto-1 protein or fragment thereof, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed, Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In one embodiment, a nucleic acid molecule of the invention comprises a nucleic acid molecule which has a nucleotide sequence complementary to the nucleotide sequence of a nucleic acid corresponding to a Cripto-3 gene or to the nucleotide sequence of a nucleic acid encoding a Cripto-3 protein. In another embodiment, a nucleic acid molecule of the invention comprises a nucleic acid molecule which has a nucleotide sequence complementary to the nucleotide sequence of a nucleic acid corresponding to a Cripto-1 gene or to the nucleotide sequence of a nucleic acid encoding a Cripto-1 protein. A preferred Cripto-3 polynucleotide has a nucleotide sequence shown in FIG. 1C (SEQ ID NO:4). A preferred Cripto-1 polynucleotide has a nucleotide sequence shown in FIG. 1B (SEQ ID NO:3). A nucleic acid molecule which is complementary to a given nucleotide sequence is one which is sufficiently complementary to the given nucleotide sequence that it can hybridize to the given nucleotide sequence thereby forming a stable duplex.

In one embodiment, a nucleic acid molecule of the invention comprises only a portion of a nucleic acid sequence, wherein the full length nucleic acid sequence comprises a Cripto-3 gene or encodes a Cripto-3 polypeptide. In another embodiment, a nucleic acid molecule of the invention comprises only a portion of a nucleic acid sequence, wherein the full length nucleic acid sequence comprises a Cripto-1 gene or encodes a Cripto-1 polypeptide. Such nucleic acid molecules are useful, for example, as probes or primers. The probe or primer typically is in the form of a substantially purified oligonucleotide. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention. In one embodiment, the oligonucleotide comprises a region of nucleotide sequence that hybridizes under stringent conditions to about 10, 11, 12, 13, 14, 15, 16, 17, 18, 19, 20, 21, 22, 23, 24, 25, 26, 27, 28, 29 or 30 or more consecutive nucleotides of a Cripto-3 polynucleotide, e.g, transcribed polynucleotide.

Nucleic acid probes based on the sequence of a Cripto-3 nucleic acid molecule can be used to detect a Cripto-3 transcribed polynucleotide, e.g., mRNA or cDNA. Nucleic acid probes based on the sequence of a Cripto-1 nucleic acid molecule can be used to detect a Cripto-1 transcribed polynucleotide, e.g., mRNA or cDNA. The probe can comprise a label group attached thereto, e.g., a radioisotope, a fluorescent compound, an enzyme, or an enzyme co-factor. Such probes can be used to specifically detect the presence of a Cripto-3 or Cripto-1 transcribed polynucleotide in a sample. Such probes can also be used, e.g., as part of a kit, to identify cells or tissues which overexpress the marker gene, e.g., transformed cells or tumor tissue, such as by measuring levels of a Cripto-3 transcribed polynucleotide in a sample of cells from a subject, e.g., detecting mRNA levels.

In one embodiment, a nucleic acid molecule of the invention is at least 7, 10, 12, 15, 20, 25, 30, 35, 40, 50, 60, 70, 80, 90, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid molecule corresponding to a Cripto-3 gene or to a nucleic acid molecule encoding a Cripto-3 protein. In another embodiment, a nucleic acid molecule of the invention is at least 7, 10, 12, 15, 20, 25, 30, 35, 40, 50, 60, 70, 80, 90, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid molecule corresponding to a Cripto-1 gene or to a nucleic acid molecule encoding a Cripto-1 protein.

As used herein, the term “hybridizes under stringent conditions” is intended to describe conditions for hybridization and washing under which nucleotide sequences at least 60%, 65%, 70%, preferably 75%, 80%, 85%, 90%, 95% or 98% identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A non-limiting example of stringent hybridization conditions includes hybridization in 6× sodium chloride/sodium citrate (SSC) at about 45° C., followed by one or more washes in 0.2×SSC, 0.1% SDS at 50-65° C. Preferred hybridization conditions allow specific detection of a Cripto-3 nucleic acid in the presence of Cripto-1 nucleic acid.

A non-limiting example of hybridization conditions particularly useful in the instant invention for the specific detection of a Cripto-3 polynucleotide in a sample comprises: (i) prehybridize in prehybridization solution (6×SSC, 5×Denhardt solution, 0.05% sodium pyrophosphate, 100 ug/ml tRNA, 0.5% SDS) at 42° C. for 1 hour; (ii) hybridize with a nucleic acid probe (e.g., a nucleic acid probe modified so as to be detectable, e.g., radioactively labeled, e.g., labeled with ³²P at the 5′-end) in hybridization solution (6×SSC, 5×Denhardt solution, 0.05% sodium pyrophosphate, 0.5% SDS) at 42° C. overnight (e.g., with radioactively labeled nucleic acid probes at 10⁶-10⁷ CPM/ml); and (iii) wash in wash solution (6×SSC containing 0.1% SDS) at 42° C. for 20 minutes, e.g., three times. One skilled in the art will recognize that optimal hybridization conditions for the specific detection of a Cripto-3 polynucleotide or a Cripto-1 polynucleotide will depend on the particular nucleic acid probe being used. Optimal hybridization conditions for a particular nucleic acid probe can be determined by one skilled in the art using no more than routine methods known in the art.

In one embodiment of the invention, nucleic acid molecules (e.g., probes or primers) hybridize to a portion of a transcribed Cripto-3 polynucleotide, which portion comprises a nucleotide sequence encoding one or more amino acids that are unique to Cripto-3 as compared to Cripto-1, such that the transcribed Cripto-3 polynucleotide is selectively detected, e.g., specifically detected when in the presence of a transcribed Cripto-1 polynucleotide. Preferably, the nucleic acid molecule (e.g., probe or primer) hybridizes weakly to a transcribed Cripto-1 polynucleotide, such that a transcribed Cripto-1 polynucleotide is weakly detected. More preferably, the nucleic acid molecule (e.g., probe or primer) does not hybridize to a transcribed Cripto-1 polynucleotide, such that a transcribed Cripto-1 polynucleotide is not detected. In another embodiment of the invention, nucleic acid molecules (e.g., probes or primers) hybridize to a portion of a transcribed Cripto-1 polynucleotide, which portion comprises a nucleotide sequence encoding one or more amino acids that are unique to Cripto-1 as compared to Cripto-3, such that the transcribed Cripto-1 polynucleotide is selectively detected, e.g., specifically detected when in the presence of a transcribed Cripto-3 polynucleotide. Preferably, the nucleic acid molecule (e.g., probe or primer) hybridizes weakly to a transcribed Cripto-3 polynucleotide, such that a transcribed Cripto-3 polynucleotide is weakly detected. More preferably, the nucleic acid molecule (e.g., probe or primer) does not hybridize to a transcribed Cripto-3 polynucleotide, such that a transcribed Cripto-3 polynucleotide is not detected.

In one embodiment, a nucleic acid molecule (e.g., primer) of the invention is at least 7, 10, 12, 15, 20, 25, 30, 35, 40, 50 or more nucleotides in length and selectively hybridizes to a nucleic acid molecule corresponding to a marker gene (e.g., Cripto-3 and/or Cripto-1) or to a nucleic acid molecule encoding a marker protein (e.g., Cripto-3 and/or Cripto-1), e.g, a transcribed polynucleotide, e.g., mRNA or cDNA, and is useful for amplification, e.g., PCR amplification, of the marker nucleic acid molecule. In one preferred embodiment, the nucleic acid molecule (e.g., primer) is capable of selectively amplifying the Cripto-3 nucleic acid molecule, e.g., Cripto-3 transcribed polynucleotide, e.g., when in the presence of a Cripto-1 nucleic acid molecule, e.g., Cripto-1 transcribed polynucleotide. Preferably, the nucleic acid molecule (e.g., primer) hybridizes weakly to a transcribed Cripto-1 polynucleotide, such that a transcribed Cripto-1 polynucleotide is weakly amplified. More preferably, the nucleic acid molecule (e.g., primer) does not hybridize to a transcribed Cripto-1 polynucleotide, such that a transcribed Cripto-1 polynucleotide is not amplified. In another preferred embodiment, the nucleic acid molecule (e.g., primer) is capable of selectively amplifying the Cripto-1 nucleic acid molecule, e.g., Cripto-1 transcribed polynucleotide, e.g., when in the presence of a Cripto-3 nucleic acid molecule, e.g., Cripto-3 transcribed polynucleotide. Preferably, the nucleic acid molecule (e.g., primer) hybridizes weakly to a transcribed Cripto-3 polynucleotide, such that a transcribed Cripto-3 polynucleotide is weakly amplified. More preferably, the nucleic acid molecule (e.g., primer) does not hybridize to a transcribed Cripto-3 polynucleotide, such that a transcribed Cripto-3 polynucleotide is not amplified.

In one embodiment, nucleic acid molecules of the invention hybridize to a portion of a transcribed Cripto-3 polynucleotide, which portion comprises a nucleotide sequence encoding one or more amino acids unique to the Cripto-3 protein as compared to the Cripto-1 protein, e.g., one or more amino acids in the Cripto-3 polypeptide sequence selected from the group consisting of V7, L68, E92 and A178. In another embodiment, nucleic acid molecules of the invention hybridize to a portion of a transcribed Cripto-1 polynucleotide, which portion comprises a nucleotide sequence encoding one or more amino acids unique to the Cripto-1 protein as compared to the Cripto-3 protein. In one preferred embodiment, the one or more amino acids unique to the Cripto-1 protein as compared to the Cripto-3 protein are selected from the group consisting of A7, P68, G92, V178, V22 and Y43. In another preferred embodiment, the one or more amino acids unique to the Cripto-1 protein as compared to the Cripto-3 protein are selected from the group consisting of A7, P68, G92 and V178.

Non-limiting examples of nucleic acid molecules useful in the instant invention for the selective detection of a Cripto-3 polynucleotide or Cripto-1 polynucleotide are set forth in Table 2. The nucleic acid molecules (probes) set forth in Table 2 correspond to the antisense or template strand of the Cripto DNA sequence. Thus, the nucleic acid molecules set forth in Table 2 are complementary to Cripto-3 or Cripto-1 mRNA (mRNA corresponds to the sense or coding strand) and are useful for hybridizing to Cripto-3 or Cripto-1 mRNA in order to detect Cripto mRNA in a sample. It will be understood by one of skill in the art that the complement of the nucleic acid molecules (probes) set forth in Table 2 correspond to the sense or coding strand of Cripto-3 or Cripto-1 DNA, and would thus be useful for hybridizing to Cripto cDNA (cDNA corresponds to the antisense or template strand) in order to detect Cripto cDNA in a sample. The nucleic acid molecules set forth in Table 2 are useful in any of the methods described herein for the specific detection of a Cripto-3 transcribed polynucleotide or the specific detection of a Cripto-1 transcribed polynucleotide in a sample. The nucleic acid molecules set forth in Table 2 are particularly useful, e.g., in Northern and Southern blot analysis for the detection of a Cripto-3 transcribed polynucleotide or Cripto-1 transcribed polynucleotide in a sample.

TABLE 2
Examples of Cripto-3-specific nucleic acid
probes and Cripto-1 specific nucleic acid
probes

| | | Specific | |
|---|---|---|---|
| | Sequence | for: | SEQ ID NO: |
| 1 | GAGAAGCGGACCATCTTCCTGCAGTC | TDGF3 | SEQ ID NO: 5 |
| 2 | AGAGAAGCGGACCATCTTCCTGCAG | TDGF3 | SEQ ID NO: 6 |
| 3 | TAAGAGAAGCGGACCATCTTCCTGC | TDGF3 | SEQ ID NO: 7 |
| 4 | GTATTCCCATGGGCAGCACACGCTG | TDGF3 | SEQ ID NO: 8 |
| 5 | CTGTATTCCCATGGGCAGCACACGC | TDGF3 | SEQ ID NO: 9 |
| 6 | GCTGTATTCCCATGGGCAGCACACG | TDGF3 | SEQ ID NO: 10 |
| 7 | GGCACAAAAGGACTCCAGCATGCAG | TDGF3 | SEQ ID NO: 11 |
| 8 | CAGGCACAAAAGGACTCCAGCATGC | TDGF3 | SEQ ID NO: 12 |
| 9 | TATAGAAAGGCAGATGCCAGCTAGC | TDGF3 | SEQ ID NO: 13 |
| 10 | CGGGTCATGAAATTTGCATAATATC | TDGF3 | SEQ ID NO: 14 |
| 11 | TACGGGTCATGAAATTTGCATAATA | TDGF3 | SEQ ID NO: 15 |
| 12 | GAGAAGCGGGCCATCTTCCTGCAGTC | TDGF1 | SEQ ID NO: 16 |
| 13 | AGAGAAGCGGGCCATCTTCCTGCAG | TDGF1 | SEQ ID NO: 17 |
| 14 | TAAGAGAAGCGGGCCATCTTCCTGC | TDGF1 | SEQ ID NO: 18 |
| 15 | GTATCCCCATGGGCGGCACACGCTG | TDGF1 | SEQ ID NO: 19 |
| 16 | CTGTATCCCCATGGGCGGCACACGCT | TDGF1 | SEQ ID NO: 20 |
| 17 | GCTGTATCCCCATGGGCGGCACACG | TDGF1 | SEQ ID NO: 21 |
| 18 | GGCACAAAAGGACCCCAGCATGCAG | TDGF1 | SEQ ID NO: 22 |
| 19 | CAGGCACAAAAGGACCCCAGCATGC | TDGF1 | SEQ ID NO: 23 |
| 20 | TATAGAAAGGCAGATGCCAACTAGC | TDGF1 | SEQ ID NO: 24 |
| 21 | CTGGTCATGAAATTTGCATGATATC | TDGF1 | SEQ ID NO: 25 |
| 22 | TACTGGTCATGAAATTTGCATGATA | TDGF1 | SEQ ID NO: 26 |

Further examples of nucleic acid molecules which are useful for the selective detection of a marker, e.g., Cripto-3 or Cripto-1, polynucleotide in a sample are nucleic acid molecules (primers) specific for a marker polynucleotide and which are suitable for amplification of the marker polynucleotide. Preferably, these nucleic acid molecules (primers) are particularly useful as primers for the PCR amplification of a marker, e.g., Cripto-3 or Cripto-1, transcribed polynucleotide in a sample. Nucleic acid molecules (primers) useful for amplification of a transcribed polynucleotide generally are used in pairs, e.g., pairs which comprise one nucleic acid molecule (primer) corresponding to the antisense or template strand of the Cripto-3 DNA sequence (e.g., the nucleotide sequence encoding Cripto-3 as shown in FIG. 1C) and a second nucleic acid molecule (primer) corresponding to the sense or coding strand of the Cripto-3 DNA sequence (e.g., the nucleotide sequence encoding Cripto-3 as shown in FIG. 1C), or “forward” and “reverse” primers. Thus, the pairs of nucleic acid molecules or primers, e.g., as set is forth in Table 3, are useful for the amplification of a portion of a transcribed polynucleotide, e.g., a Cripto-3 mRNA (wherein mRNA corresponds to the sense or coding strand) and/or Cripto-3 cDNA (wherein cDNA corresponds to the antisense or template strand), which is flanked by the two nucleic acid primers, in order to specifically detect a transcribed polynucleotide, e.g., mRNA or cDNA, in a sample. Nonlimiting examples of such nucleic acid molecules (primers) are set forth in Table 3. While the nucleic acid molecules set forth in Table 3 are presented as specific pairs of forward and reverse primers, one of skill in the art will recognize that other combinations of forward and reverse primers, e.g., primers as set forth in Table 3, may be used to specifically amplify a Cripto-3 or Cripto-1 transcribed polynucleotide. It will be further understood by one of skill in the art that while the nucleic acid molecules set forth in Table 3 are particularly useful in amplification methods of the invention, e.g., PCR, e.g., quantitative PCR, these nucleic acid molecules are also useful in any of the other detection methods described herein for the specific detection of a Cripto-3 transcribed polynucleotide or for the specific detection of a Cripto-1 transcribed polynucleotide in a sample.

TABLE 3

Examples of Cripto-3-specific nucleic acid primers and
Cripto-1-specific nucleic acid primers

| | | | Size of | |
|---|---|---|---|---|
| Specific | | | PCR | |
| for: | Sequences of primers: | Primer: | product: | SEQ ID NO: |
| TDGF3 | GCGTGTGCTGCCCATGGGA | Forward | 431 bp | SEQ ID NO: 27 |
| | CGGGTCATGAAATTTGCATA | Reverse | | SEQ ID NO: 28 |
| TDGF3 | GACTGCAGGAAGATGGTCCGCTTC | Forward | 211 bp | SEQ ID NO: 29 |
| | TTCCCATGGGCAGCACACGCT | Reverse | | SEQ ID NO: 30 |
| TDGF3 | GCGTGTGCTGCCCATGGGAATAC | Forward | 97 bp | SEQ ID NO: 31 |
| | GCAGGCACAAAAGGACTCCAG | Reverse | | SEQ ID NO: 32 |
| TDGF3 | GCGTGTGCTGCCCATGGGAATAC | Forward | 349 bp | SEQ ID NO: 31 |
| | GCAGATGCCAGCTAGCATAAAAG | Reverse | | SEQ ID NO: 33 |
| TDGF3 | GCTAGCTGCCATCTGCCTTTC | Forward | 99 bp | SEQ ID NO: 34 |
| | ACGGGTCATGAAATTTGCATAA | Reverse | | SEQ ID NO: 35 |
| TDGF1 | GCTACGACCTTCTGGGGAAAACG | Forward | 776 bp | SEQ ID NO: 36 |
| | CTGGTCATGAAATTTGCATG | Reverse | | SEQ ID NO: 37 |
| TDGF1 | GACTGCAGGAAGATGGCCCGCTTC | Forward | 211 bp | SEQ ID NO: 38 |
| | TCCCCATGGGCGGCACACGCT | Reverse | | SEQ ID NO: 39 |
| TDGF1 | GCGTGTGCCGCCCATGGGGATAC | Forward | 97 bp | SEQ ID NO: 40 |
| | GCAGGCACAAAAGGACCCCAG | Reverse | | SEQ ID NO: 41 |
| TDGF1 | GCGTGTGCCGCCCATGGGGATAC | Forward | 349 bp | SEQ ID NO: 40 |
| | GCAGATGCCAACTAGCATAAAAG | Reverse | | SEQ ID NO: 42 |
| TDGF1 | GCTAGTTGGCATCTGCCTTTC | Forward | 99 bp | SEQ ID NO: 43 |
| | CTGGTCATGAAATTTGCATGA | Reverse | | SEQ ID NO: 44 |

The invention also includes molecular beacon nucleic acid molecules having at least one region which is complementary to a Cripto-3 or Cripto-1 nucleic acid molecule as described herein, such that the molecular beacon is useful for quantitating the presence of the Cripto-3 or Cripto-1 nucleic acid molecule in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,976,930.

In one embodiment, a nucleic acid molecule can contain sequences which naturally flank the nucleic acid (i.e., sequences located at the 5′ and 3′ ends of the nucleic acid) in the genomic DNA of the organism from which the nucleic acid is derived. In various embodiments, the isolated nucleic acid molecule can contain about 100 kB, 50 kB, 25 kB, 15 kB, 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. For example, in various embodiments, the nucleic acid molecules of the invention contain temporal and spatial regulatory elements (e.g., elements that restrict the expression of the markers of the invention to a specific tissue or to a specific developmental stage), that are proximal or 5′ to the initiation signal, e.g., the initiating ATG codon. Moreover, a nucleic acid molecule can be substantially free of other cellular material or culture medium when produced by recombinant techniques, or substantially free of chemical precursors or other chemicals when chemically synthesized.

It will be understood by those skilled in the art that the invention further encompasses nucleic acid molecules that differ, due to degeneracy of the genetic code, from the nucleotide sequence of nucleic acid molecules encoding a marker protein, e.g., Cripto-3 and/or Cripto-1, and thus encode the same protein. It will further be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a Cripto-3 polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

The skilled artisan will further appreciate that sequence changes can be introduced into a nucleic acid molecule of the invention by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a marker polypeptide, e.g., a Cripto-3 or Cripto-1 polypeptide that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 80%, 90%, 95%, or 98% identical to the amino acid sequence of one of the proteins which correspond to the markers of the invention.

A nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

Nucleic acid molecules of the invention corresponding to temporal and spatial regulatory elements, e.g., temporal and spatial promotors, of a marker gene can be used to construct recombinant expression vectors. The identification of temporal and spatial regulatory elements can be performed by creating recombinant expression vectors containing nucleic acid molecules with putative temporal and spatial regulatory elements operably linked to sites of inducible recombination, such as, for example, lox sites, e.g., loxP sites, and optionally further operably linked to a reporter sequence, such as, for example, LacZ, GFP, and EGFP. Such recombinant expression vectors can be used to generate transgenic animals, the cells of which can subsequently be examined for temporal and spatial restriction of the reporter sequence to identify nucleic acid molecules of the invention corresponding to temporal and spatial regulatory elements.

Such transgenic animals as described above are not only useful for identifying spatial and temporal regulatory elements, but are also useful for studying the function and/or activity of the marker polypeptide of the invention, for identifying and/or evaluating modulators of the marker polypeptide activity, as well as in pre-clinical testing of therapeutics or diagnostic agents. Furthermore, such animals are useful for the investigation of the effect, e.g., physiological effect, of a temporal and spatial restriction of a gene of interest. For example, a transgene may cause lethality due to the requirement of the gene at a particular point in development. However, the same transgene under the control of a spatially and/or temporally regulated promoter may be induced subsequent to the point in time that loss of the gene causes lethality and/or in a specific tissue that does not cause lethality. Alternatively, a gene that is ubiquitously expressed or expressed at a low or undetectable level in normal cells, e.g., cells not afflicted with a disease, disorder, or condition, may be preferentially overexpressed or misexpressed in a disease, disorder, or condition, such as, for example, cancer. For example, Cripto-1 protein is expressed at a low level or is undetectable in many tissues of the adult, but has been shown to be overexpressed specifically in cells of the mammary gland as well as in many cancers. Similarly, as described herein, Cripto-3 transcribed polynucleotide is expressed at a low level or is undetectable in many normal adult tissues, but is overexpressed specifically in many cancers. Operably linking Cripto-3 to a spatially restricted promoter of the invention, e.g., a mammary gland-specific promoter, and further operably linking an inducible promoter, will allow controlled expression, e.g., inducible expression; of Cripto-3 in specific cell types, e.g., cells of the mammary gland, in order to more closely model a proliferative disease, disorder, or condition for the study of the progression, maintenance, and/or response to treatment of the proliferative disease, disorder, or condition.

### IV. ISOLATED PROTEINS AND ANTIBODIES

(i). Proteins

One aspect of the invention pertains to isolated Cripto proteins, e.g., Cripto-3, as well as polypeptide fragments suitable for use as immunogens to raise antibodies directed against a Cripto-3 polypeptide. In one embodiment, the Cripto-3 polypeptide or fragment thereof can be isolated from cells or tissue sources by an appropriate purification scheme using standard protein purification techniques. In another embodiment, the Cripto-3 polypeptide or fragment thereof is produced by recombinant DNA techniques. Alternative to recombinant expression, the Cripto-3 polypeptide or fragment thereof can be synthesized chemically using standard peptide synthesis techniques.

An “isolated” or “purified” protein or fragment thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

A preferred Cripto-3 polypeptide has an amino acid sequence shown in FIG. 1A (SEQ ID NO:2). Other useful proteins are substantially identical (e.g., at least about 40%, preferably 50%, 60%, 70%, 80%, 90%, 95%, or 99%) to this sequence and retain the functional activity of the naturally-occurring Cripto-3 protein yet differ in amino acid sequence due to mutagenesis or to natural allelic variation.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

In one embodiment of the invention, an isolated Cripto-3 polypeptide, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a Cripto-3 protein comprises at least 8 (preferably 10, 15, 20, 25 or 30 or more) amino acid residues of the amino acid sequence of Cripto-3, and encompasses an epitope of the protein such that an antibody raised against the peptide forms a specific immune complex with a Cripto-3 protein. Preferably, the antigenic peptide of a Cripto-3 protein encompasses an epitope of the Cripto-3 protein such that an antibody raised against the peptide selectively forms an immune complex with Cripto-3 protein and does not form an immune complex with a Cripto-1 protein.

Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions. The antigenic peptide may correspond to an entire domain, such as the extracellular domain, intracellular domains, the EGF-like domain, the cys-rich domain, the receptor binding domain, and the like. The extracellular domain of Cripto-3 spans from about amino acid residue 1 to about amino acid residue 158 of the mature Cripto-3 protein. The EGF-like domain of Cripto-3 spans from about amino acid residue 75 to about amino acid residue 112 of the mature Cripto-3 protein. The cys-rich domain spans from about amino acid residue 114 to about amino acid residue 150 of the mature Cripto-3 protein. Epitopes in Cripto-3 may comprise linear or nonlinear spans of amino acid residues. In one embodiment, the epitope is an epitope formed in the conformationally native Cripto-3 protein versus a denatured Cripto-3 protein.

In one embodiment of the invention, the antigenic peptide of a Cripto-3 protein encompasses an epitope of the Cripto-3 protein comprised in the extracellular domain. Preferably, epitopes encompassed by the antigenic peptide are regions of a Cripto-3 protein comprising one or more of the amino acid residues which are unique to Cripto-3 as compared to Cripto-1, e.g., amino acid residues in the amino acid sequence of the Cripto-3 polypeptide which are different from the amino acid residues at the corresponding amino acid positions of the amino acid sequence of the Cripto-1 polypeptide. Amino acid residues in the Cripto-3 polypeptide which are different from the corresponding amino acid residues of the Cripto-1 polypeptide include amino acids V7, L68, E92 and A178 of Cripto-3. A preferred epitope encompassed by the antigenic peptide is a region of Cripto-3 comprising at least one amino acid selected from the group consisting of V7, L68, E92 and A178. In a related embodiment, the antigenic peptide of a Cripto-3 protein comprises a portion of the Cripto-3 protein, wherein the secondary structure or conformation of the portion is unique to Cripto-3 as compared to Cripto-1, e.g., owing to the presence of one or more amino acids unique to Cripto-3 as compared to Cripto-1, e.g., V7, L68, E92 and A178.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

The invention also provides chimeric or fusion proteins corresponding to a Cripto-3 polypeptide. As used herein, a “chimeric protein” or “fusion protein” comprises all or part (preferably a biologically active part) of a Cripto-3 polypeptide operably linked to a heterologous polypeptide (i.e., a polypeptide other than the Cripto-3 polypeptide). Within the fusion protein, the term “operably linked” is intended to indicate that the Cripto-3 polypeptide and the heterologous polypeptide are fused in-frame to each other. The heterologous polypeptide can be fused to the amino-terminus or the carboxyl-terminus of the Cripto-3 polypeptide.

One useful fusion protein is a GST fusion protein in which a Cripto-3 polypeptide is fused to the carboxyl terminus of GST sequences. Such fusion proteins can facilitate the purification of a recombinant Cripto-3 polypeptide.

In another embodiment, the fusion protein contains a heterologous signal sequence at its amino terminus. For example, the native signal sequence of a Cripto-3 polypeptide can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al. supra) and the protein A secretory signal (Pharmacia Biotech; Piscataway, N.J.).

In yet another embodiment, the fusion protein is an immunoglobulin fusion protein in which all or part of a Cripto-3 polypeptide is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a Cripto-3 polypeptide. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a Cripto-3 polypeptide in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al. supra). Moreover, many expression vectors are commercially available that already encode a fusion moiety (e.g., a GST polypeptide). A nucleic acid encoding a Cripto-3 polypeptide can be cloned into such an expression vector such that the fusion moiety is linked in-frame to the Cripto-3 polypeptide.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of a Cripto-3 polypeptide. Such variants have an altered amino acid sequence which can function as either agonists (mimetics) or as antagonists. Variants can be generated by mutagenesis, e.g., discrete point mutation or truncation. Preferred variants maintain one or more of the specific amino acids of Cripto-3 which are different from those of Cripto-1, e.g., variants that are not altered at one or more of amino acids V7, L68, E92 and A178 of Cripto-3. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a Cripto-3 polypeptide can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al. 1993, Protein Engineering 6(3):327-331).

It is further contemplated that the methods described herein may be used to produce antibodies that form a specific immune complex with Cripto-1, e.g., do not form an immune complex with Cripto-3. Preferably, the antigenic peptide of a Cripto-1 protein encompasses an epitope of the Cripto-1 protein such that an antibody raised against the peptide selectively forms an immune complex with Cripto-1 protein and does not form an immune complex with a Cripto-3 protein. Preferably, epitopes encompassed by the antigenic peptide are regions of a Cripto-1 protein comprising one or more of the amino acid residues which are unique to Cripto-1 as compared to Cripto-3, e.g., amino acid residues in the polypeptide sequence of Cripto-1 which are different from the amino acid residues in the corresponding amino acid positions of the polypeptide sequence of Cripto-3. Amino acid residues in the Cripto-1 polypeptide which are different from the corresponding amino acids within the Cripto-3 polypeptide include amino acids A7, P68, G92, V178, V22 and Y43 of Cripto-1. A preferred epitope encompassed by the antigenic peptide is a region of Cripto-1 comprising at least one of amino acids A7, P68, G92 and V178. In a related embodiment, the antigenic peptide of a Cripto-1 protein comprises a portion of the Cripto-1 protein, wherein the secondary structure or conformation of the portion is unique to Cripto-1 as compared to Cripto-3, e.g., owing to the presence of one or more amino acids unique to Cripto-1 as compared to Cripto-3, e.g., A7, P68, G92 and V178.

(ii). Antibodies

Another aspect of the invention pertains to antibodies directed against a Cripto-3 polypeptide. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a Cripto-3 polypeptide of the invention. A molecule which specifically binds to a Cripto-3 polypeptide is a molecule which binds the Cripto-3 polypeptide, but does not substantially bind other molecules in a sample, e.g., a biological sample, which naturally contains the polypeptide. Preferably, a molecule which specifically binds to a Cripto-3 polypeptide does not substantially bind a Cripto-1 polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a Cripto-3 polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al. 1994, Bio/technology 12:899-903).

An antibody, antibody derivative, or fragment thereof, which specifically binds a Cripto-3 polypeptide which is modulated in a proliferative disorder, e.g., cancer, may be used to inhibit activity of Cripto-3, and therefore may be administered to a subject to treat, inhibit, or prevent a proliferatie disorder, e.g., cancer, in the subject. Furthermore, conjugated antibodies may also be used to treat, inhibit, or prevent cancer in a subject. Conjugated antibodies, preferably monoclonal antibodies, or fragments thereof, are antibodies which are joined to drugs, toxins, or radioactive atoms, and used as delivery vehicles to deliver those substances directly to cancer cells. The antibody, e.g., an antibody which specifically binds a Cripto-3 polypeptide, is administered to a subject and binds the marker, thereby delivering the toxic substance to the afflicted cell, minimizing damage to normal cells in other parts of the body.

Conjugated antibodies are also referred to as “tagged,” “labeled,” or “loaded.” Antibodies with chemotherapeutic agents attached are generally referred to as chemolabeled. Antibodies with radioactive particles attached are referred to as radiolabeled, and this type of therapy is known as radioimmunotherapy (RIT). Aside from being used to treat cancer, radiolabeled antibodies can also be used to detect areas of cancer spread in the body. Antibodies attached to toxins are called immunotoxins.

Immunotoxins are made by attaching toxins (e.g., poisonous substances from plants or bacteria) to monoclonal antibodies. Immunotoxins may be produced by attaching monoclonal antibodies to bacterial toxins such as diphtherial toxin (DT) or pseudomonal exotoxin (PE40), or to plant toxins such as ricin A or saporin.

An antibody directed against a Cripto-3 polypeptide (e.g., a monoclonal antibody) can be used to isolate the polypeptide by standard techniques, such as affinity chromatography or immunoprecipitation. Moreover, such an antibody can be used to detect the polypeptide (e.g., in a cellular lysate or cell supernatant) in order to evaluate the level and pattern of expression of the polypeptide. The antibodies can also be used diagnostically to monitor protein levels in tissues or body fluids (e.g. in an ovary-associated body fluid) as part of a clinical testing procedure, e.g., to, for example, determine the efficacy of a given treatment regimen (e.g., efficacy of an anti-Cripto antibody therapy). Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, O-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

It is further contemplated that any of the methods described herein may be used for the preparation of a molecule, e.g., an antibody, e.g., a monoclonal antibody, that specifically binds to Cripto-1 polypeptide, e.g., does not bind to a Cripto-3 polypeptide. A molecule which specifically binds to a Cripto-1 polypeptide is a molecule which binds the Cripto-1 polypeptide, but does not substantially bind other molecules in a sample, e.g., a biological sample, which naturally contains the polypeptide. Preferably, a molecule which specifically binds to a Cripto-1 polypeptide does not substantially bind a Cripto-3 polypeptide.

### IV. DETECTION AND DIAGNOSTIC ASSAYS

The present invention relates to detection assays or diagnostic assays for determining the amount, structure, and/or activity of polypeptides or nucleic acids corresponding to a marker of the invention, e.g., Cripto-3 and/or Cripto-1, in a sample. Such detection assays are useful for determining the phenotype of a tumor, e.g., whether a tumor is a Cripto-3 or Cripto-1 expressing tumor. Such assays are also useful for assessing whether a cell (e.g., a cell from a patient sample) is transformed, e.g., for diagnosing cancer, and for assessing whether a patient is a suitable candidate for an anti-Cripto antibody therapy. In a preferred embodiment, the expression level of a marker of the invention, e.g., Cripto-3, can be assayed as a method for assessing whether a cell, e.g., a cell from a patient sample, is transformed. The expression level of a marker of the invention, e.g., Cripto-3, can further be assayed as a method for diagnosis of a proliferative disease, disorder or condition, e.g., cancer, or risk for developing a proliferative disease, disorder or condition. Additionally, the expression level of a marker of the invention, e.g., Cripto-3, can be assayed as a method for assessing whether a patient is suitable for an anti-Cripto antibody therapy.

1. Methods for Detection of Gene Expression

Expression of a marker of the invention may be assessed by any of a wide variety of well known methods for detecting expression of a transcribed polynucleotide or protein. Non-limiting examples of such methods include nucleic acid hybridization methods, nucleic acid reverse transcription methods, and nucleic acid amplification methods, immunological methods for detection of secreted, cell-surface, cytoplasmic, or nuclear proteins, protein purification methods and protein function or activity assays.

In preferred embodiments, expression of a particular marker, e.g., Cripto-3 and/or Cripto-1, is characterized by a measure of gene transcript (e.g. mRNA or cDNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see Sambrook et al. supra). For example, one method for evaluating the presence, absence, or quantity of cDNA involves a Southern transfer. Briefly, the mRNA is isolated (e.g. using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such detection assays or diagnostic and prognostic assays involves preparing a sample or reaction mixture that contains a marker and a probe (e.g., a nucleic acid probe or primer), under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe (e.g., nucleic acid probe or primer), when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al. U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe (e.g., nucleic acid probe or primer) to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al. ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In particular embodiments, the level of transcribed polynucleotide, e.g., mRNA, corresponding to the marker can be determined either by in situ or by in vitro formats in a biological sample using methods known in the art. The term “biological sample” is intended to include tissues, cells, biological fluids and isolates thereof, isolated from a subject, as well as tissues, cells and fluids present within a subject, e.g. tumor cells.

The expression detection methods of the invention can use isolated RNA, e.g., mRNA, or cDNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155). For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

The isolated transcribed polynucleotide, e.g., mRNA or cDNA, corresponding to a marker of the invention can be used in hybridization or amplification assays that include, but are not limited to, Southern or Northern analyses, polymerase chain reaction analyses and probe arrays.

One diagnostic method for the detection of mRNA or cDNA levels involves contacting the isolated mRNA or cDNA with a nucleic acid molecule (probe) that can hybridize to the mRNA or cDNA encoded by the marker being detected. The nucleic acid probe can be, for example, a full-length cDNA, or a portion thereof, such as an oligonucleotide of at least 7, 10, 15, 20, 25, 30, 40, 50, 75, 100, 150, 200, 250, 300, 400, 500, 600, 700, 800, 900 or 1000 nucleotides or more in length and sufficient to specifically hybridize under stringent conditions to a transcribed polynucleotide, e.g., mRNA or cDNA, encoding a marker of the present invention, e.g., Cripto-3 and/or Cripto-1. In one embodiment, the nucleic acid probe selectively hybridizes to a transcribed Cripto-3 polynucleotide such that the nucleic acid probe does not hybridize to a transcribed Cripto-1 polynucleotide. In another embodiment, the nucleic acid probe selectively hybridizes to a transcribed Cripto-1 polynucleotide such that the nucleic acid probe does not hybridize to a transcribed Cripto-3 polynucleotide. Other suitable nucleic acid probes or primers for use in the methods of the invention are described herein. Hybridization of a transcribed polynucleotide, e.g., mRNA or cDNA, with the probe or primer indicates that the marker in question is being expressed.

In one format, the transcribed polynucleotide, e.g., mRNA or cDNA, is immobilized on a solid surface and contacted with a probe, for example by running the isolated mRNA or cDNA on an agarose gel and transferring the mRNA or cDNA from the gel to a membrane, such as nitrocellulose. In an alternative format, the probe(s) are immobilized on a solid surface and the mRNA or cDNA is contacted with the probe(s), for example, in an Affymetrix gene chip array. A skilled artisan can readily adapt known mRNA and/or cDNA detection methods for use in detecting the level of mRNA or cDNA encoded by the markers of the present invention.

The probes can be full length or less than the full length of the nucleic acid sequence encoding the protein. Shorter probes are empirically tested for specificity. Preferably nucleic acid probes are 10, 15, 20 bases or longer in length. (See, e.g., Sambrook et al. for methods of selecting nucleic acid probe sequences for use in nucleic acid hybridization.) Visualization of the hybridized portions allows the qualitative determination of the presence or absence of the mRNA or cDNA.

In one embodiment of the invention, determining the level of a transcribed polynucleotide corresponding to a marker of the invention, e.g., Cripto-3 and/or Cripto-1, involves the process of amplification of the transcribed polynucleotide, followed by the detection of the amplified molecules using techniques well known to those of skill in the art. Methods for nucleic acid amplification are known to those skilled in the art, and include, but are not limited to, e.g., PCR, rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al. 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method known to those skilled in the art. Fluorogenic rtPCR may also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. A preferred amplification method of the invention involves PCR, e.g., rtPCR.

In general, nucleic acid molecules that can be used as amplification primers are from about 10 to 50 (e.g., 10, 11, 12, 13, 14, 15, 16, 17, 18, 19, 20, 21, 22, 23, 24, 25, 26, 27, 28, 29, 30, 35, 40, 45, 50 or more) nucleotides in length and flank a region of about 25, 50, 75, 100, 200, 300, 400, 500, 750, 10000, 20000 or more nucleotides in length. Under appropriate conditions and with appropriate reagents, such primers permit the amplification of a nucleic acid molecule comprising the nucleotide sequence flanked by the primers.

In one embodiment, the nucleic acid molecule (primer) selectively hybridizes to a transcribed Cripto-3 polynucleotide such that the primer selectively amplifies the transcribed Cripto-3 polynucleotide and does not amplify a transcribed Cripto-1 polynucleotide. One example of such a nucleic acid molecule (primer) is a nucleic acid molecule (primer) which hybridizes to a portion of the transcribed Cripto-3 polynucleotide, which portion comprises nucleotides encoding an amino acid which is unique to Cripto-3 as compared to Cripto-1, e.g., an amino acid residue in the Cripto-3 polynucleotide sequence which is different from the corresponding amino acid in the Cripto-1 polynucleotide sequence, e.g., an amino acid selected from the group consisting of: V7, L68, E92 and A178. In another embodiment, the nucleic acid molecule (primer) selectively hybridizes to a transcribed Cripto-1 polynucleotide such that the primer selectively amplifies the transcribed Cripto-1 polynucleotide and does not amplify a transcribed Cripto-3 polynucleotide. One example of such a nucleic acid molecule (primer) is a nucleic acid molecule (primer) which hybridizes to a portion of the transcribed Cripto-1 polynucleotide, which portion comprises nucleotides encoding one or more amino acids which are unique to Cripto-1 as compared to Cripto-3, e.g., one or more amino acid residues in the Cripto-1 polynucleotide sequence which are different from the corresponding amino acids in the Cripto-3 polynucleotide sequence, e.g., one or more amino acids selected from the group consisting of: A7, P68, G92, V178, V22 and Y43. In a preferred embodiment, the one or more amino acids unique to the Cripto-1 protein as compared to the Cripto-3 protein are selected from the group consisting of A7, P68, G92 and V178.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal, non-cancerous sample, or between samples from different sources.

Alternatively, the expression level can be provided as a relative expression level. To determine a relative expression level of a marker, the level of expression of the marker is determined for 1, 2, 3, 4, 5, 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

It is further contemplated that the expression level of Cripto-3 can be provided as a relative expression level to the expression level of Cripto-1 in the sample. In one embodiment, the invention provides a method of assessing whether a patient is a suitable candidate for an anti-Cripto antibody therapy, the method comprising comparing the level of expression of a TDGF3 gene in a patient sample, e.g., tumor sample, and the level of expression of a TDGF1 gene in the patient sample, wherein a higher level of expression of the TDGF3 gene in the patient sample, as compared to the level of expression of the TDGF1 gene in the patient sample, is an indication that the patient is a suitable candidate for an anti-Cripto antibody therapy. Preferably, the samples used in the baseline determination will be from cancer cells or normal cells of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the transformed state, e.g., cancer state.

Because the compositions, kits, and methods of the invention rely on detection of a difference in expression levels of a marker of the invention, it is preferable that the level of expression of the marker is significantly greater than the minimum detection limit of the method used to assess expression in at least one of normal cells and cancerous cells.

In another preferred embodiment, expression of a marker is assessed by preparing genomic DNA or mRNA/cDNA (i.e. a transcribed polynucleotide) from cells in a subject sample, and by hybridizing the genomic DNA or mRNA/cDNA with a reference polynucleotide which is a complement of a polynucleotide comprising the marker, and fragments thereof cDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g. single nucleotide polymorphisms, deletions, etc.) of a marker of the invention may be used to detect occurrence of a mutated marker in a subject.

In another embodiment, a combination of methods to assess the expression of a marker is utilized.

2. Methods for Detection of Expressed Protein

The activity or level of a marker protein of the invention, e.g., Cripto-3 and/or Cripto-1, can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like. A skilled artisan can readily adapt known protein/antibody detection methods for use in determining whether cells express a marker of the present invention.

A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In a preferred embodiment, the antibody is labeled, e.g. a radio-labeled, chromophore-labeled, fluorophore-labeled, or enzyme-labeled antibody). In another embodiment, an antibody derivative (e.g. an antibody conjugated with a substrate or with the protein or ligand of a protein-ligand pair (e.g. biotin-streptavidin)), or an antibody fragment (e.g. a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies may be directly labeled or alternatively may be subsequently detected using labeled antibodies (e.g., labeled sheep anti-mouse antibodies) that specifically bind to the anti-polypeptide.

In a more preferred embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one preferred embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g. as biotin, to which a third labeled molecule can specifically bind, such as enzyme-labeled streptavidin.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 11: 1401-1406, and Akerstrom (1985) J. Immunol., 135: 2589-2542).

As indicated above, immunoassays for the detection and/or quantification of a polypeptide can take a wide variety of formats well known to those of skill in the art.

Preferred immunoassays for detecting a polypeptide are either competitive or noncompetitive. Noncompetitive immunoassays are assays in which the amount of captured analyte is directly measured. In one preferred “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In one particularly preferred embodiment, the antibody is immobilized on a solid substrate. The amount of polypeptide bound to the antibody may be determined either by measuring the amount of polypeptide present in a polypeptide/antibody complex, or alternatively by measuring the amount of remaining uncomplexed polypeptide. The amount of polypeptide may be detected by providing a labeled polypeptide.

The assays of this invention are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

Antibodies for use in the various immunoassays described herein, can be produced as described above.

In another embodiment, level (activity) is assayed by measuring the enzymatic activity of the gene product. Methods of assaying the activity of an enzyme are well known to those of skill in the art.

In vivo techniques for detection of a biomarker protein include introducing into a subject a labeled antibody directed against the protein. For example, the antibody can be labeled with a radioactive marker whose presence and location in a subject can be detected by standard imaging techniques.

It will be appreciated that subject samples, e.g., a sample containing tissue or cells, e.g., tumor tissue or cells, e.g., whole blood, serum, plasma, buccal scrape, saliva, spinal fluid, cerebrospinal fluid, urine, stool, may contain cells therein, particularly when the cells are cancerous, and, more particularly, when the cancer is metastasizing, and thus may be used in the methods of the present invention. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the level of expression of the marker in the sample. Thus, the compositions, kits, and methods of the invention can be used to detect expression of markers corresponding to proteins having at least one portion which is displayed on the surface of cells which express it. It is a simple matter for the skilled artisan to determine whether the protein corresponding to any particular marker comprises a cell-surface protein. For example, immunological methods may be used to detect such proteins on whole cells, or well known computer-based sequence analysis methods (e.g. the SIGNALP program; Nielsen et al., 1997, Protein Engineering 10: 1-6) may be used to predict the presence of at least one extracellular domain (i.e. including both secreted proteins and proteins having at least one cell-surface domain). Expression of a marker corresponding to a protein having at least one portion which is displayed on the surface of a cell which expresses it may be detected without necessarily lysing the cell (e.g. using a labeled antibody which binds specifically with a cell-surface domain of the protein).

The invention also encompasses kits for detecting the presence of a polypeptide or nucleic acid corresponding to a marker of the invention in a biological sample, e.g., a sample containing tissue or cells, e.g., tumor tissue or cells, or biological fluids, e.g., whole blood, serum, plasma, buccal scrape, saliva, spinal fluid, cerebrospinal fluid, urine, stool. Such kits can be used to determine if a cell is transformed. Such kits can also be used to assess whether a subject is a suitable candidate for an anti-Cripto antibody therapy. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or a nucleic acid molecule (probe or primer) which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For nucleic acid molecule-based kits, the kit can comprise, for example: (1) an nucleic acid molecule (probe), e.g., a detectably labeled nucleic acid molecule, which hybridizes to a nucleic acid sequence encoding a polypeptide corresponding to a marker of the invention or (2) a pair of nucleic acid molecules (primers) useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

This invention is further illustrated by the following examples which should not be construed as limiting. The contents of all references, figures, tables, Appendices, Accession Numbers, patents and published patent applications cited throughout this application are hereby incorporated by reference.

### EXAMPLES

### Example 1

### TDGF3 (Cripto-3) Gene Expression Analysis

### A. Materials and Methods

Human genomic DNA and RNA Samples. Healthy control human genomic DNAs were purchased from Sigma (HRC1 panel) and Coriell Cell Repositories. Human cancer genomic DNA and RNAs were purchased from Biochain Institute Inc. (Hayward, Calif. 94545), or prepared from human tissues (Asterand, Detroit, Mich. 48202) and cell cultures. All human carcinoma cell lines were obtained from the American Type Culture Collection except for KM20L2, which was obtained from the NCI-DCTD tumor repository. DNAs were prepared with DNeasy kits (Qiagen, Valencia, Calif.). RNAs were prepared with Trizol followed up with RNeasy kit (Qiagen, Valencia, Calif.). To eliminate any residual genomic DNA contamination in the “genomic DNA free” RNAs, 1 μg of each RNA sample was digested by 1 unit of DNase I (Invitrogen) for 15 or 30 minute at room temperature prior to cDNA synthesis. cDNAs were checked for gDNA contamination by PCR with Cripto-1 intron specific oligo primers (CGCTTACAGGAATTGCCCTTGC (SEQ ID NO:45); AND CAGACCCAAGCTATCGCAGC (SEQ ID NO; 46)).

cDNA preparation and TA cloning. cDNA was synthesized with SMART cDNA synthesis kit (ED Clontech, CA) according to the manufacturer's protocol. 0.3 μg of total RNA which was free of genomic DNA was used for each sample. cDNA from each sample was used as template to amplify full length or a fragment of TDGF 1 and Cripto-3 cDNA, using the following oligo primer pairs common to both genes:

(SEQ ID NO: 47)
GGCTGAGTCTCCAGCTCAAGG (FL, forward)
and
(SEQ ID NO: 48)
GTATTTCTGGAAATAGGTCAATGTCG (FL, reverse);
(SEQ ID NO: 47)
GGCTGAGTCTCCAGCTCAAGG (Partial, forward)
and
(SEQ ID NO: 49)
TGTGATTTGGATCATGGCCA (Partial, reverse).

The PCR products were cloned into pCR2.1-TOPO vector (Invitrogen) and multiple isolates from each tissue sample were sequenced.
Genotyping with pyrosequencer. Target DNA fragments were PCR amplified from 20 ng genomic DNA in 20 μl reaction volume containing 0.4 μl Titanium Taq DNA polymerase (BD Biosciences, Palo Alto, Calif.). PCR conditions were as following: Initial denature at 95° C. for 1 minute, denature at 94° C. for 45 seconds, annealing at 64° C. for 45 seconds, extension at 72° C. for 0.5 minute, and repeat for 38 cycles. Sequences of the primers were as follows:

(SEQ ID NO: 50)

CTCATGTTTGACTTCCTCTTC (Forward);

(SEQ ID NO: 51)

CATCGAAGTCAGGCAGTTCTTAC (reverse, biotinylated at
5′ end);

(SEQ ID NO: 52

GATCATGGCCATTTCTAAAG (sequencing primer for
V/A22);

(SEQ ID NO: 53)

GAATTTGCTCGTCCATCTCGGGGA (sequencing primer for
Y/D43).

Approximately 3-8 μl of PCR product was used for genotyping on PSQ96 HS instruments (Biotage, Uppsala, Sweden) according to the protocols suggested by the manufacture.

Cripto-1 and Cripto-3 specific PCRs. PCR conditions were as follows: Initial denature at 95° C. for 1 minute, denature at 94° C. for 45 seconds, annealing at 64° C. (Cripto-3 specific) or 66° C. (Cripto-1 specific) for 45 seconds, extension at 72° C. for 1 minutes, and repeat for 35 cycles Sequences of the primers were as follows:

| Forward oligonucleotide for Cripto-1: | |
|---|---|
| GCTACGACCTTCTGGGGAAAACG; | (SEQ ID NO: 36) |
| Reverse oligonucleotide for Cripto-1: | |
| CTGGTCATGAAATTTGCATG; | (SEQ ID NO: 37) |
| Forward oligonucleotide for Cripto-3: | |
| GCGTGTGCTGCCCATGGGA; | (SEQ ID NO: 27) |
| Reverse oligonucleotide for Cripto-3: | |
| CGGGTCATGAAATTTGCATA. | (SEQ ID NO: 28) |

The expected sizes of the PCR products were 776 bp for the Cripto-1 fragment and 431 bp for the Cripto-3 fragment.

FACS detection of Cripto proteins Cells were incubated with 10 μg/ml of anti-Cripto antibody and stained with anti-mouse IgG PE conjugated secondary antibody. Approximately 10,000 cells were analyzed by flow cytometry using FACS Calibur and FlowJo software.

### B. Results

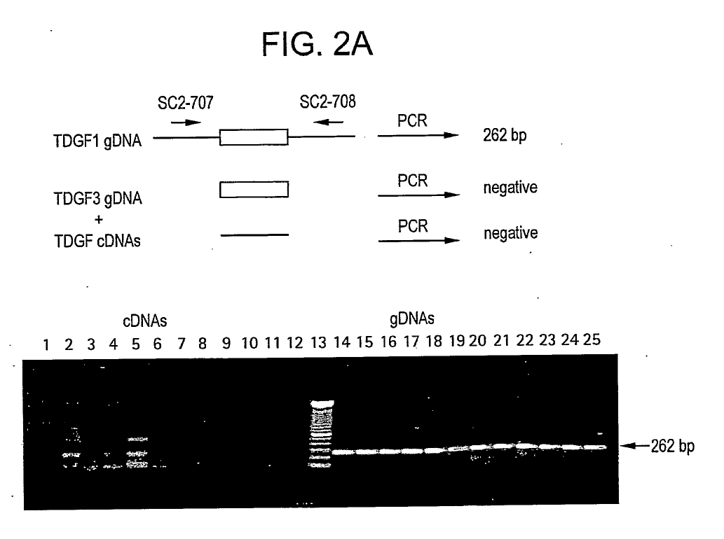

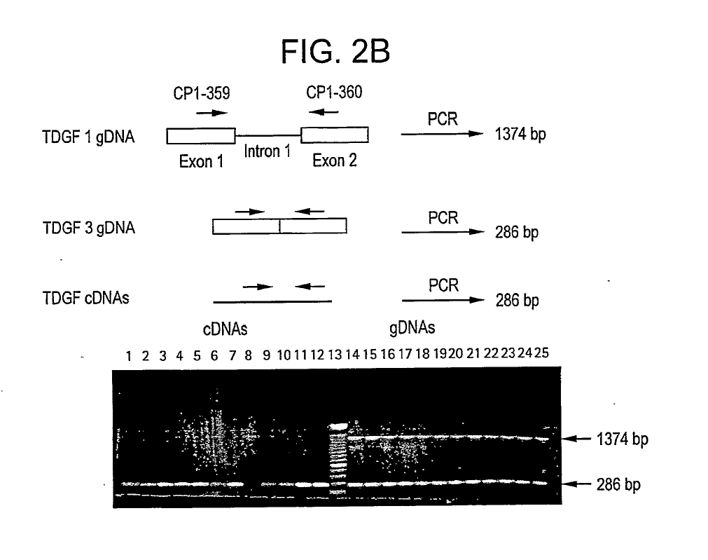

Cripto-3 gene expression. To investigate whether the Cripto-3 gene is expressed in human tumor tissues and tumor cell lines, Cripto cDNA from human tumor tissue samples and tumor cell lines was amplified with primers common to Cripto-1 and Cripto-3 (FIG. 2B). The PCR products were then cloned and multiple clones from each sample were sequenced. Since Cripto-3 is an intronless gene, its genomic coding sequence is identical to the cDNA sequence. Thus, any genomic DNA contaminating RNA preparations could be amplified and mistaken as Cripto-3 cDNAs. To eliminate genomic DNA (gDNA) contamination, RNA preparations were rigorously treated with DNase I prior to reverse transcription, and the cDNAs were confirmed to be free of genomic DNA contamination by carrying out PCR with primers specific to Cripto-1 introns (FIG. 2). Surprisingly, all isolates of the cDNA clones from cancer samples were derived from the Cripto-3 gene, and Cripto-3 cDNAs were also found in 5 out of 8 cancer cell lines tested (Table 4).

TABLE 4
Summary of cDNA fragment clones

| | # cDNA Fr. | # of Cripto-1 | # of Cripto-3 |
|---|---|---|---|
| Tissues | isolates | isolates | isolates |
| Lung tumor #1 | 10 | 0 | 10 |
| Lung tumor #2 | 7 | 0 | 7 |
| Lung tumor #3 | 8 | 0 | 8 |
| Lung tumor #4 | 9 | 0 | 9 |
| Breast tumor #1 | 9 | 0 | 9 |
| Breast tumor #2 | 9 | 0 | 9 |
| Breast tumor #3 | 9 | 0 | 9 |
| Breast tumor #4 | 8 | 0 | 8 |
| Colon tumor #1 | 9 | 0 | 9 |
| Colon tumor #2 | 9 | 0 | 9 |
| Colon tumor #3 | 9 | 0 | 9 |
| Colon tumor #4 | 9 | 0 | 9 |
| Normal Breast | 8 | 0 | 8 |
| Normal Lung | 12 | 7 | 5 |
| Normal Lung | 12 | 0 | 12 |
| Colo205 | 8 | 8 | 0 |
| NCCIT | 9 | 9 | 0 |
| H727 | 8 | 8 | 0 |
| GEO | 10 | 9 | 1 |
| BT474 | 10 | 0 | 10 |
| MCF7 | 10 | 0 | 10 |
| LS174T | 10 | 9 | 1 |
| H69 | 8 | 0 | 8 |

To control for the possibility that the TA cloned cDNA fragments originated from mRNA fragments that do not have coding potential, full-length TDGF cDNAs from additional cancer tissue samples were PCR amplified with primers common to Cripto-1 and Cripto-3. Full-length cDNA clone isolates from all TDGF positive cancer samples were found to be derived from the Cripto-3 gene (Table 5). Cripto-3 cDNAs were also found in one normal breast, two normal colon and three normal lung samples (Table 5). Cripto-1 cDNAs were only amplified from healthy tissues, including three of six breast samples and two of six lung samples (Table 5). Only one normal lung sample yielded cDNA clones derived from both Cripto-1 and Cripto-3 genes. (Table 5 and Table 4).

TABLE 5
Summary of full-length Cripto clones

| | | # of Cripto | # of | | |
|---|---|---|---|---|---|
| | Total # | FL PCR | FL isolates | # of | # of |
| Tissues | of samples | positive | sequenced | Cripto-1 | Cripto-3 |
| Normal | 6 | 3 | 70 | 70 | 0 |
| Breast | | 1 | 5 | 0 | 5 |
| Normal | 6 | 2 | 30 | 0 | 30 |
| Colon | | | | | |
| Normal | 6 | 1 | 8 | 8 | 0 |
| Lung | | 2 | 43 | 0 | 43 |
| | | 1 | 21 | 12 | 9 |
| Breast | 12 | 8 | 111 | 0 | 111 |
| Tumor | | | | | |
| Colon | 12 | 3 | 35 | 0 | 35 |
| Tumor | | | | | |
| Lung | 12 | 5 | 55 | 0 | 55 |
| Tumor | | | | | |

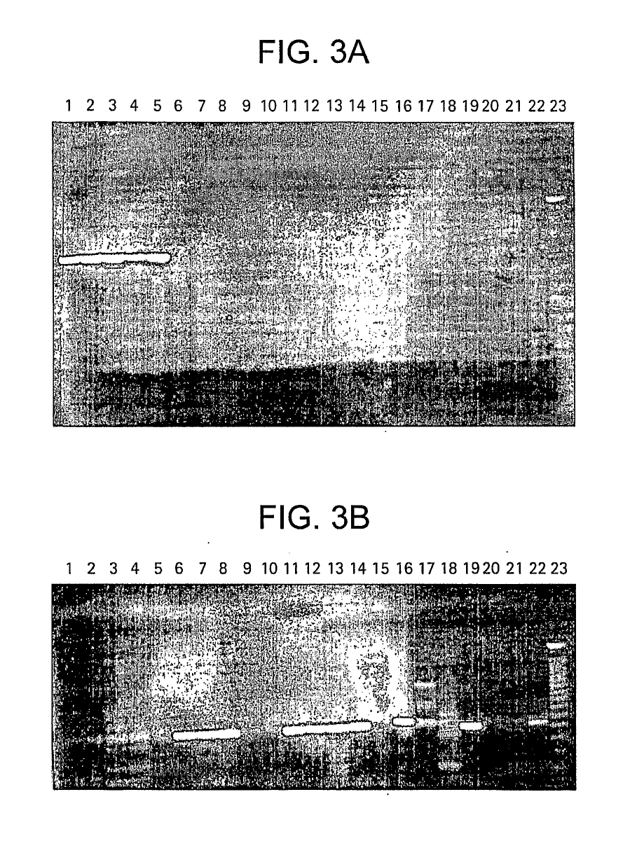

Transcript-specific PCR. It was possible that PCR using primers common to Cripto-1 and Cripto-3 was generating data biased toward one gene transcript over the other. To obtain independent experimental data for TDGF expression, the fixed nucleotide differences between the two genes was capitalized on to perform transcript-specific PCR on the same set of cDNAs as above. There are four fixed amino acid differences between the two proteins: Alanine 7 (A7), Proline 68 (P68), Glycine 92 (G92), and Valine 178 (V178) in Cripto-1, as compared to Valine 7 (V7), Leucine 68 (L68), Glutamic acid 92 (E92) and Alanine 178 (A178) in Cripto-3. Primers specific to the nucleotide sequences encoding one or more of these unique amino acids were used to specifically amplify Cripto-1 or Cripto-3 transcript. As shown in FIG. 3A, three normal breast and two normal lung samples expressed Cripto-1. In contrast, all the cancer samples tested in this experiment were either positive for Cripto-3 (FIG. 3B) or had a negative RT-PCR result.

SAGE database search. Next, the public SAGE database (see the web site at www.ncbi.nlm.nih.gov/projects/SAGE) was searched for independent evidence of Cripto-3 expression. The TDGF1-specific tag used in the search was the sequence TAATTCTACCAAGGTCT. The Cripto-3 specific tag used in the search was the sequence CTCTTCAGAA. Two non-overlapping sets of SAGE libraries were positive for either Cripto-1 or Cripto-3 tag (Among the ten Cripto-1 tag positive libraries, nine were from ES cells and one was from fetal brain. Four of the five Cripto-3 positive libraries were from cancer samples. These data further support the conclusion that Cripto-3 is expressed in cancer tissues.

Affymetrix gene expression data. Next, further independent evidence supporting expression of Cripto-3 rather than Cripto-1 in tumors was derived from Affymetrix gene expression data. Probe set 40386_r_at on the human U95Av2 Affymetrix chip is annotated as Cripto-1-specific. However, only one of the 16 probes in this probe set is specific for Cripto-1, while all of the other probes are common to both Cripto-1 and Cripto-3. “Cripto-1” expression was detected in 28 of 42 human malignant colon samples with this probe set. The Cripto-1-specific oligonucleotide probe consistently had the lowest intensity of all probes in the set, as the signal was almost always “absent.” (p<0.01). The lack of signal from the Cripto-1-specific probe in these experiments is consistent with the Cripto signal being derived from Cripto-3 rather than Cripto-1 message.

Sequence variation analysis. Finally, to test whether the observed cDNA sequences might have derived from polymorphisms or mutations in Cripto-1, sequence variation in Cripto-1 and Cripto-3 was analyzed in DNA from 96 healthy controls, 72 cancer samples and 32 cell lines. For Cripto-1, two previously reported SNPs, rs11130097 and rs2293025, showed allele frequencies similar to that from healthy populations reported in dbSNP. Cripto-1 was polymorphic at two sites which were monomorphic in Cripto-3. In particular, Cripto-1 was observed to be polymorphic at amino acid residue 22, e.g., V/A 22 (T/C at nucleotide position 312), and at amino acid residue 43, e.g., Y/D 43 (T/G at nucleotide position 374). Cripto-3 was found to be monomorphic at these two positions, where the specific amino acid in the Cripto-3 sequence is the same as the amino acid at that position in one of the two variants in the Cripto-1 sequence, e.g., A22 and D43. Thus the amino acids V22 and Y43 are unique to Cripto-1. The estimated allele frequency for V22 is about 47% in Caucasian (94 individuals genotyped) and 57% in African American (86 individuals genotyped). The allele frequency for D43 is 4% in Caucasian (94 individuals genotyped) and 1% in African Americans (96 individuals genotyped). Otherwise, no SNPs or mutations were found on sites at which Cripto-1 and Cripto-3 differ. For Cripto-3, the entire coding sequence was sequenced in these 204 samples and no nucleotide variation was observed; all sequences were identical to those observed for Cripto-3 cDNA clones described herein.

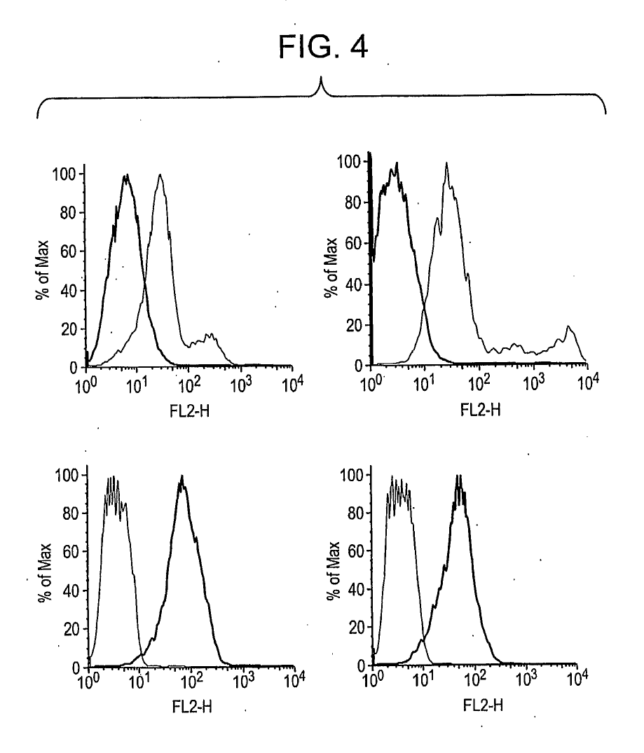

Cripto-3 protein expression. The next question addressed was whether Cripto-3 mRNA is translated in cells and the resulting Cripto-3 protein is transported to the cell surface. Two anti-Cripto antibodies, B3.F6 and A6.C12, were used to measure Cripto-3 protein by Fluorescence Activated Cell Sorting (FACS). BT474, a cell line from which only Cripto-3 cDNA could be PCR amplified successfully, showed the same pattern of staining as the cell line NCCIT, which is positive for Cripto-1 RT-PCR only (FIG. 4A). Similar results were obtained from a cell line T47D (negative for endogenous Cripto expression) which had been transfected with a plasmid expressing either Cripto-1or Cripto-3 (FIG. 4B). These results demonstrated that the Cripto-3 gene was not only transcribed, but that Cripto-3 protein was translated and transported to the cell surface in these cells.

Regulation of Cripto-3 expression. The observed expression of Cripto-3 in some cancers as provided herein raises the question of how this gene is de-regulated in these cells. Possible mechanisms by which Cripto-3 expression may be deregulated in cancer include, but are not limited to: (i) amplification of the Cripto-3 locus; (ii) Mutations in promoter/regulatory region of Cripto-3; (iii) up- or down-regulation of transcription factor(s) that regulate Cripto-3 expression; and (iv) changes in methylation status of the Cripto-3 promoter. Mining of genome-wide expression profiles revealed several genes whose expression pattern is positively correlated with TDGF, including ASCL2, DHCR7, EPHB3, GPSM2, NOX1, C13orf23, and NUFIP1. In particular, ASCL2 encodes a transcription factor that binds to clusters of E-Box sequence fragments. There are 6 E-Boxes within the 300 nucleotide region upstream of the Cripto-3 transcription start site, while there is only one E-Box in the 300 nucleotide fragment upstream of the Cripto-1 transcription start site. These data suggest a possible link between the ASCL2 transcription factor and Cripto-3 gene expression. Further genomic analysis can shed light on how Cripto-3 gene expression is modulated in cancers, providing more information on how to target Cripto or its interacting proteins for cancer treatment.

### C. Discussion

There are 6 amino acid differences between the Cripto-1 and Cripto-3 reference sequences, four of which are fixed differences and two of which are at sites that are polymorphic in Cripto-1. As indicated in FIG. 1, the fixed amino acid differences are: A7V, P68L, G92E, V178A (Cripto1 sequence indicated first). While the functional implications of these amino acid differences are not presently known, the non-conservative substitution of proline with leucine at position 68 is noteworthy. It is also notable that, among all of the TDGF pseudogenes in the human genome, Cripto-3 is the only pseudogene that has maintained its open reading frame. Moreover, no mutations or SNPs were found in the samples tested herein. The existence of an intronless TDGF pseudogene on the chimpanzee X chromosome (refseq contig NW122118.1) indicates that Cripto-3 is at least as old as the human species. Therefore, it appears that the Cripto-3 gene is either in a portion of the genome with reduced variation, and/or has been subjected to purifying selection. Intense purifying selection on Cripto-3 is consistent with Cripto-3 having a functional role in addition to any role the gene plays in cancer.

Prior to the instant invention, all published work on TDGF expression has used methods which do not distinguish between Cripto-1 and Cripto-3. Results from IHC, Northern blot analysis and genome-wide oligonucleotide chips have been attributed to Cripto-1 simply because Cripto-3 bears some hallmarks of a pseudogene, such as a lack of introns. However, the expression of intronless genes is not rare; it is estimated that about 5% of human genes are intronless.¹⁵ The only publicly available data with sufficient sequence specificity to distinguish Cripto-1 from Cripto-3 is SAGE, but the data indicating Cripto-3 expression in cancers has been overlooked.

Applicants are the first to provide evidence that the presumed pseudogene Cripto-3 is a functional intronless gene and that Cripto-3 is the only Cripto gene expressed at detectable levels in a significant number of TDGF positive cancer tissues. It is clear that Cripto-1 plays key roles during early embryonic development and that engineered over-expression of Cripto-1 is oncogenic. The observations that Cripto-3 is expressed in cancer tissues and that Cripto-3 has a similar coding sequence to Cripto-1 strongly suggest that Cripto-3 expression is also oncogenic. Thus while Cripto-1 appears to have evolved to play important roles during embryonic development and mammary gland development, it is predicted that expression of either Cripto-1 or Cripto-3 at high level in adult tissues is oncogenic.

### Example 2

### TDGF3 (Cripto-3) Functions in Cripto-Nodal Signaling Assay

### A. Materials and Methods

Mouse teratocarcinoma F9 Cripto−/− cells, null for mouse Cripto, (2×10⁵ cells/well in a 24 welled plate) were transfected using Lipofectamine (Invitrogen) with 50 ng of (n2)₇-luciferase reporter construct, 100 ng forkhead activin signal transducer (FAST) transcription factor, and 100 ng full length human Cripto-1 or human Cripto-3 expression plasmid. 48 hours following transfection, the cells were lysed with LucLite (Perkin Elmer) and the luciferase activity was measured in a luminometer (Perkin Elmer).

### B. Results

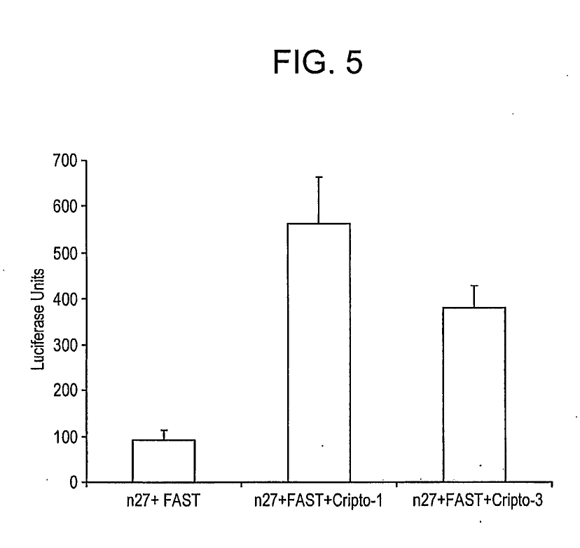

Human Cripto-1 and human Cripto-3 were tested for the ability to signal through Nodal in a FAST transcription factor dependent (n2)₇-luciferase reporter assay. Activity was assessed in a mouse F9 derived embryonal carcinoma cell line gene targeted for inactivation of the mouse Cripto locus, (F9 Cripto −/−). These cells contain endogenous Nodal but are null for Cripto and thus are null for Cripto-dependent signaling. There was a 4 to 6 fold increase of luciferase activity in Cripto-3 and Cripto-1 transfected cells when compared with control (FIG. 5). These results indicate that human Cripto-1 and human Cripto-3 are both capable of signalling through Nodal.

### REFERENCES

Adkins H B, Bianco C, Schiffer S G, Rayhorn P, Zafari M, Cheung A E, Orozco O, Olson D, De Luca A, Chen L L, Miatkowski K, Benjamin C, Normanno N, Williams K P, Jarpe M, LePage D, Salomon D, Sanicola M (2003) Antibody blockade of the Cripto CFC domain suppresses tumor cell growth in vivo. J Clin Invest 112: 575-87
Brocke K S N-Y G, Gehring N H, Hentze M W, Kulozik A E (2002) The human intronless melanocortin 4-receptor gene is NMD insensitive. Hum Mol Genet. 11: 331-5
Ciardiello F, Dono R, Kim N, Persico M G, Salomon D S (1991a) Expression of cripto, a novel gene of the epidermal growth factor gene family, leads to in vitro transformation of a normal mouse mammary epithelial cell line. Cancer Res 51: 1051-4
Ciardiello F, Kim N, Saeki T, Dono R, Persico M G, Plowman G D, Garrigues J, Radke S, Todaro G J, Salomon D S (1991b) Differential expression of epidermal growth factor-related proteins in human colorectal tumors. Proc Natl Acad Sci USA 88: 7792-6
de la Cruz J M, Bamford R N, Burdine R D, Roessler E, Barkovich A J, Donnai D, Schier A F, Muenke M (2002) A loss-of-function mutation in the CFC domain of TDGF1 is associated with human forebrain defects. Hum Genet 110: 422-8
Gritsman K, Zhang J, Cheng S, Heckscher E, Talbot W S, Schier A F (1999) The EGF-CFC protein one-eyed pinhead is essential for nodal signaling. Cell 97: 121-32
Minchiotti G, Manco G, Parisi S, Lago C T, Rosa F, Persico M G (2001) Structure-function analysis of the EGF-CFC family member Cripto identifies residues essential for nodal signalling. Development 128: 4501-10
Normanno N, De Luca A, Bianco C, Maiello M R, Carriero M V, Rehman A, Wechselberger C, Arra C, Strizzi L, Sanicola M, Salomon D S (2004a) Cripto-1 overexpression leads to enhanced invasiveness and resistance to anoikis in human MCF-7 breast cancer cells. J Cell Physiol 198: 31-9
Normanno N, De Luca A, Maiello M R, Bianco C, Mancino M, Strizzi L, Arra C, Ciardiello F, Agrawal S, Salomon D S (2004b) CRIPTO-1: a novel target for therapeutic intervention in human carcinoma. Int J Oncol 25: 1013-20
Saloman D S, Bianco C, Ebert A D, Khan N I, De Santis M, Normanno N, Wechselberger C, Seno M, Williams K, Sanicola M, Foley S, Gullick W J, Persico G (2000) The EGF-CFC family: novel epidermal growth factor-related proteins in development and cancer. Endocr Relat Cancer 7: 199-226
Scognamiglio B B G, Cassano C, Tucci M, Montuori N, Dono R, Lembo G, Barra A, Lago C T, Viglietto G, Rocchi M, Persico M G. (1999) Assignment of human teratocarcinoma derived growth factor (TDGF) sequences to chromosomes 2q37, 3q22, 6p25 and 19q13.1. Cytogenet Cell Genet. 84: 220-4.
Shen M M (2003) Decrypting the role of Cripto in tumorigenesis. J Clin Invest 112: 500-2
Strizzi L, Bianco C, Normanno N, Salomon D (2005) Cripto-1: a multifunctional modulator during embryogenesis and oncogenesis. Oncogene. 2005 24: 5731-41.
Sun Y S L, Raafat A, Hirota M, Bianco C, Feigenbaum L, Kenney N, Wechselberger C, Callahan R, Salomon D S. (2005) Overexpression of human Cripto-1 in transgenic mice delays mammary gland development and differentiation and induces mammary tumorigenesis. Am J. Pathol. 167: 585-597
Xing P X, Hu X, Pietersz G A, Hosick H L, McKenzie I F (2004) Cripto: a novel target for antibody-based cancer immunotherapy. Cancer Res 64: 4018-23

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for detecting the presence of a TDGF3 polynucleotide or portion thereof in a sample, the method comprising the steps of:
a) contacting the sample with a nucleic acid molecule which selectively hybridizes to a transcribed TDGF3 polynucleotide, wherein the transcribed TDGF3 polynucleotide comprises the coding region of the TDGF3 gene; and
b) determining whether the nucleic acid molecule binds to the polynucleotide in the sample, to thereby detect the presence of the TDGF3 polynucleotide or portion thereof in the sample.

2. (canceled)

3. (canceled)

4. (canceled)

5. (canceled)

6. (canceled)

7. (canceled)

8. (canceled)

9. The method of claim 1, wherein the at least one nucleic acid molecule hybridizes to a portion of the transcribed TDGF3 polynucleotide, which portion comprises nucleotides within the TDGF3 coding region encoding an amino acid selected from the group consisting of: V7, L68, E92 and A178.

10. The method of claim 9, wherein the at least one nucleic acid molecule comprises a sequence selected from the group of sequences set forth in Table 2 and Table 3.

11. A method for detecting the presence of a TDGF3 polypeptide or portion thereof in a sample, the method comprising the steps of:
a) contacting the sample with an antibody, antibody derivative or antibody fragment which selectively binds to a TDGF3 polypeptide; and
b) determining whether the antibody, antibody derivative or antibody fragment binds to the polypeptide in the sample, to thereby detect the presence of the TDGF3 polypeptide or portion thereof in the sample.

12. (canceled)

13. (canceled)

14. (canceled)

15. The method of claim 11, wherein the antibody, antibody derivative or antibody fragment binds to an epitope comprising an amino acid selected from the group consisting of: V7, L68, E92 and A178.

16. The method of claim 1 or claim 11, wherein the sample comprises a tumor tissue sample.

17. The method of claim 16, wherein the tumor is selected from the group consisting of a breast tumor, colon tumor and lung tumor.

18. The method of claim 1 or claim 11, wherein the sample is a body fluid.

19. The method of claim 18, wherein the body fluid is selected from the group consisting of blood, lymph, ascetic fluid, gynecological fluid, cystic fluid and urine.

20. (canceled)

21. (canceled)

22. (canceled)

23. A method of assessing whether a cell is transformed, comprising comparing:
a) the level of expression of a TDGF3 gene in a test cell, and
b) the level of expression of a TDGF3 gene in a control non-transformed cell,
wherein a higher level of expression of the TDGF3 gene in the test cell as compared to the level in the control non-transformed cell is an indication that the test cell is transformed.

24. The method of claim 23, wherein the level of expression of the TDGF3 gene in the test cell and in the control cell is assessed by detecting the presence in the test cell and in the control cell of a transcribed polynucleotide or portion thereof, wherein the transcribed polynucleotide comprises the coding region of the TDGF3 gene, using at least one nucleic acid molecule which selectively hybridizes to the TDGF3 coding region.

25. (canceled)

26. (canceled)

27. (canceled)

28. (canceled)

29. (canceled)

30. (canceled)

31. The method of claim 24, wherein the at least one nucleic acid molecule hybridizes to a portion of transcribed polynucleotide corresponding to the TDGF3 coding region which spans the nucleotides encoding an amino acid selected from the group consisting of: V7, L68, E92 and A178.

32. The method of claim 31, wherein the at least one nucleic acid molecule comprises a sequence selected from the group of sequences set forth in Table 2 and Table 3.

33. The method of claim 23, wherein the level of expression of the TDGF3 gene in the test cell and in the control cell is assessed by detecting the presence in the test cell and in the control cell of a protein encoded by the TDGF3 gene using an antibody, antibody derivative or antibody fragment that specifically binds with the protein.

34. (canceled)

35. (canceled)

36. (canceled)

37. The method of claim 33, wherein the antibody, antibody derivative or antibody fragment binds to an epitope comprising one or more amino acids selected from the group consisting of: V7, L68, E92 and A178.

38. A kit for assessing the presence in a sample of transformed cells, the kit comprising a reagent selected from the group consisting of an antibody, antibody derivative, or fragment thereof, that specifically binds with a TDGF3 protein and a nucleic acid molecule that selectively hybridizes with a TDGF3 transcribed polynucleotide.

39. (canceled)

40. A method of assessing whether a patient is a suitable candidate for an anti-Cripto antibody therapy, the method comprising comparing:
a) the level of expression of a TDGF3 gene in a patient sample, and
b) the level of expression of a TDGF3 gene in a control non-cancer sample,
wherein a higher level of expression of the TDGF3 gene in the patient sample, as compared to the control non-cancer sample, is an indication that the patient is a suitable candidate for an anti-Cripto antibody therapy.

41. The method of claim 40, wherein the level of expression of the TDGF3 gene in the sample is assessed by detecting the presence in the sample of a transcribed polynucleotide or portion thereof, wherein the transcribed polynucleotide comprises the coding region of the TDGF3 gene, using at least one nucleic acid molecule which selectively hybridizes to the TDGF3 coding region.

42. (canceled)

43. (canceled)

44. (canceled)

45. (canceled)

46. (canceled)

47. (canceled)

48. The method of claim 41, wherein the at least one nucleic acid molecule hybridizes to a portion of transcribed polynucleotide corresponding to the TDGF3 coding region which spans the nucleotides encoding an amino acid selected from the group consisting of: V7, L68, E92 and A178.

49. The method of claim 48, wherein the at least one nucleic acid molecule comprises a sequence selected from the group of sequences set forth in Table 2 and Table 3.

50. The method of claim 40, wherein the nucleic acid molecule selectively hybridizes with a portion of a transcribed TDGF3 polynucleotide under stringent hybridization conditions.

51. The method of claim 40, wherein the level of expression of the TDGF3 gene in the sample is assessed by detecting the presence in the sample of a protein encoded by the TDGF3 gene using an antibody, antibody derivative or antibody fragment that specifically binds with the protein.

52. (canceled)

53. (canceled)

54. (canceled)

55. The method of claim 51, wherein the antibody, antibody derivative or antibody fragment binds to an epitope comprising one or more amino acids selected from the group consisting of: V7, L68, E92 and A178.

56. The method of claim 40, wherein the patient sample comprises a tumor tissue sample.

57. The method of claim 56, wherein the tumor is selected from the group consisting of a breast tumor, colon tumor and lung tumor.

58. The method of claim 40, wherein the patient sample is a body fluid.

59. The method of claim 58, wherein the body fluid is selected from the group consisting of blood, lymph, ascetic fluid, gynecological fluid, cystic fluid and urine.

60. The method of claim 40, wherein the level of expression of TDGF3 gene in the patient sample differs from the level of expression of the TDGF3 gene in a control non-cancer sample by a factor of at least about 2-fold.

61. The method of claim 40, wherein the level of expression of TDGF3 gene in the patient sample differs from the level of expression of the TDGF3 gene in a control non-cancer sample by a factor of at least about 5-fold.

62. The method of claim 40, wherein the TDGF3 gene is not expressed in the control non-cancer sample.

63. A kit for assessing whether a patient is a suitable candidate for an anti-Cripto antibody therapy, the kit comprising a reagent selected from the group consisting of an antibody, antibody derivative, or fragment thereof, that specifically binds with a TDGF3 protein and a nucleic acid molecule that selectively hybridizes with a TDGF3 transcribed polynucleotide.

64. (canceled)

65. A method of selecting a composition for inhibiting cellular transformation in a cell, the method comprising:
a) obtaining a sample comprising cells, and
b) separately maintaining aliquots of the sample in the presence of a plurality of test compositions;
c) comparing expression of a TDGF3 gene in each of the aliquots;
d) selecting one of the test compositions which induces a lower level of expression of the TDGF3 gene in the aliquot containing that test composition, relative to other test compositions.

66. A method of assessing the carcinogenic potential of a test compound, the method comprising:
a) maintaining separate aliquots of mammalian cells in the presence and absence of the test compound; and
c) comparing expression of a TDGF3 gene in each of the aliquots;
d) wherein a greater level of expression of the TDGF3 gene in the aliquot maintained in the presence of the test compound, relative to the aliquot maintained in the absence of the test compound, is an indication that the test compound possesses carcinogenic potential.

67. A method of making an isolated monoclonal antibody useful for specifically detecting the presence of a TDGF3 polypeptide or portion thereof in a sample, the method comprising:
isolating a TDGF3 polypeptide or portion thereof;
immunizing a mammal using the isolated polypeptide;
isolating splenocytes from the immunized mammal;
fusing the isolated splenocytes with an immortalized cell line to form hybridomas; and
screening individual hybridomas for production of an antibody which specifically binds with the TDGF3 polypeptide; and
isolating the antibody produced by the hybridoma, to thereby isolate a monoclonal antibody useful for specifically detecting the presence of a TDGF3 polypeptide or portion thereof in a sample.

68. A monoclonal antibody produced by the method of claim 67.

69. A method for detecting the presence of a TDGF1 polynucleotide or portion thereof in a sample, the method comprising the steps of:
a) contacting the sample with a nucleic acid molecule which selectively hybridizes to a transcribed TDGF1 polynucleotide, wherein the transcribed TDGF1 polynucleotide comprises the coding region of the TDGF1 gene; and
b) determining whether the nucleic acid molecule binds to the polynucleotide in the sample, to thereby detect the presence of the TDGF1 polynucleotide or portion thereof in the sample.

70. (canceled)

71. (canceled)

72. (canceled)

73. (canceled)

74. (canceled)

75. (canceled)

76. (canceled)

77. The method of claim 69, wherein the at least one nucleic acid molecule hybridizes to a portion of the transcribed TDGF1 polynucleotide, which portion comprises nucleotides within the TDGF1 coding region encoding an amino acid selected from the group consisting of: A7, P68, G92, V178, V22 and Y43.

78. The method of claim 77, wherein the at least one nucleic acid molecule comprises a sequence selected from the group of sequences set forth in Table 2 and Table 3.

79. A method for detecting the presence of a TDGF1 polypeptide or portion thereof in a sample, the method comprising the steps of:
a) contacting the sample with an antibody, antibody derivative or antibody fragment which selectively binds to a TDGF3 TDGF1 polypeptide; and
b) determining whether the antibody, antibody derivative or antibody fragment binds to the polypeptide in the sample, to thereby detect the presence of the TDGF1 polypeptide or portion thereof in the sample.

80. (canceled)

81. (canceled)

82. (canceled)

83. The method of claim 79, wherein the antibody, antibody derivative or antibody fragment binds to an epitope comprising an amino acid selected from the group consisting of: A7, P68, G92, V178, V22 and Y43.

84. The method of claim 69 or claim 79, wherein the patient sample comprises a tumor tissue sample.

85. The method of claim 84, wherein the tumor is selected from the group consisting of a breast tumor, colon tumor and lung tumor.

86. The method of claim 69 or claim 79, wherein the patient sample is a body fluid.

87. The method of claim 86, wherein the body fluid is selected from the group consisting of blood, lymph, ascetic fluid, gynecological fluid, cystic fluid and urine.

88. (canceled)

89. A kit for detecting the expression of a TDGF1 gene in a sample, the kit comprising a reagent selected from the group consisting of a nucleic acid molecule that selectively hybridizes with a TDGF1 transcribed polynucleotide and an antibody, antibody derivative, or fragment thereof, that specifically binds with a TDGF1 polypeptide or portion thereof.

90. (canceled)

91. An isolated nucleic acid molecule for specifically detecting a TDGF1 polynucleotide, wherein the nucleic acid molecule is selected from the group of sequences set forth in Table 2 and Table 3.

92. An isolated nucleic acid molecule for specifically detecting a TDGF3 polynucleotide, wherein the nucleic acid molecule is selected from the group of sequences set forth in Table 2 and Table 3.
